(19) United States
(12) Patent Application Publication
(10) Pub. No.: US 2021/0068713 A1
(43) Pub. Date: Mar. 11, 2021

(54) DETECTING SWIMMING ACTIVITIES ON A WEARABLE DEVICE

(71) Applicant: Apple Inc., Cupertino, CA (US)

(72) Inventors: Gunes DERVISOGLU, Santa Clara, CA (US); Einav YOGEV, Raanana (IL); Barak SAGIV, Kochav-Yair (IL); Alexander Singh ALVARADO, Sunnyvale, CA (US); Stephen P. JACKSON, San Francisco, CA (US); Karthik JAYARAMAN RAGHURAM, Mounain View, CA (US); Hung A. PHAM, Oakland, CA (US)

(73) Assignee: Apple Inc., Cupertino, CA (US)

(21) Appl. No.: 17/015,965

(22) Filed: Sep. 9, 2020

Related U.S. Application Data
(60) Provisional application No. 62/897,829, filed on Sep. 9, 2019.

(51) Int. Cl. A61B 5/11; A61B 5/00; A61B 5/0205

(52) U.S. Cl. CPC A61B 5/1123; A61B 5/681; A61B 5/0205; A61B 5/02416; A61B 5/742; A61B 5/7285; A61B 5/7264

(57) ABSTRACT

Disclosed embodiments include wearable devices and techniques for detecting swimming activities, classifying user motion, detecting water submersion, and monitoring performance during swimming activities. By accurately and promptly detecting swimming activities and automatically distinguishing between different swimming stroke type performed during a swimming activity, the disclosure enables wearable devices to accurately calculate user performance information when users forget to start and/or stop recording swimming activities. In various embodiments, swimming activity detection techniques may improve the selectivity of motion based methods of identifying swimming activities identification by confirming motion analysis with water immersion and pressure data analysis that detects when the wearable device is submerged in water.

### CROSS REFERENCE TO RELATED APPLICATIONS

This application claims the benefit of U.S. Provisional Application Ser. No. 62/897,829 filed Sep. 9, 2019, the entire contents of which is hereby incorporated by reference.

### FIELD

The present disclosure relates generally to detecting swimming activities using a wearable device.

### BACKGROUND

A wearable device may be worn on the hand, wrist, or arm of a person when swimming. It may be desirable to track swimming activities by a user to promote exercise and for other health related reasons. Detecting the start and end points of a swimming activity is an essential component of accurately tracking swimming activities.

### SUMMARY

In one aspect, disclosed herein are computer implemented methods for improving performance of a wearable device while recording a swimming activity, the methods including receiving motion data of a user from one or more motion sensors of the wearable device. Embodiments may also include receiving pressure data from a pressure sensor of the wearable device. Embodiments may also include detecting, by a processor circuit of the wearable device, a start of the swimming activity, the detecting the start of the swimming activity including determining, by the processor circuit using the motion data, rotational data expressed in a frame of reference based on the motion data. Embodiments may also include classifying, by the processor circuit a user's arm swing as a swim stroke motion based on the rotational data and the motion data. Embodiments may also include detecting, by the processor circuit, the swim stroke motion in the motion data for a first predetermined period of time. Embodiments may also include confirming, by processor circuit, the user is swimming based on the pressure data. Embodiments may also include determining, by the processor circuit one or more swimming metrics for the swimming activity in response to detecting the start of the swimming activity.

In some embodiments, the confirming the user is swimming based on the pressure data may include, sampling, by the processor circuit, a plurality of pressure signals from the pressure data. Embodiments may also include continuously comparing, by the processor circuit, the plurality of pressure signals to a high pressure threshold. Embodiments may also include detecting, by the processor circuit, at least one pressure signal that exceeds the high pressure threshold.

Embodiments may also include detecting, by the processor circuit, an end of the swimming activity based on the motion data and the rotational data by, determining, by the processor circuit, the user's arm swing does not include the swim stroke motion for a second predetermined period of time. Embodiments may also include determining, by the processor circuit, a user heading at multiple time points during the swimming activity based on the rotational data. Embodiments may also include continuously calculating, by the processor circuit, a change in user heading during the swimming activity. Embodiments may also include determining, by the processor circuit, the user heading is not changing on a periodic basis.

Embodiments may also include confirming the end of the swimming activity based on pressure data. Embodiments may also include sampling, by the processor circuit, a plurality of pressure signals from the pressure data. Embodiments may also include continuously comparing, by the processor circuit, the plurality of pressure signals to a dry pressure threshold. Embodiments may also include detecting, by the processor circuit, at least one pressure signal included in the plurality of pressure signals is below the dry pressure threshold.

Embodiments may also include determining, by the processor circuit, a user heading from the rotational data at a first time point and a second time point. Embodiments may also include calculating, by the processor circuit, a change in device heading at the second time point relative to the first time point. Embodiments may also include comparing, by the processor circuit, the change in user heading to a change in heading threshold.

Embodiments may also include in response to determining the change in user heading exceeds the change in heading threshold, determining, by the processor circuit, a user is performing a turn during a swimming activity. Embodiments may also include in response to determining the change in user heading is below the change in heading threshold, confirming, by the processor circuit, an end of the swimming activity.

In some embodiments, the classifying the user's arm swing as a swimming motion may also include, determining, by the processor circuit, a moment arm for the user's arm swing during a fundamental period. Embodiments may also include comparing, by the processor circuit, the moment arm to a moment arm threshold. Embodiments may also include detecting, by the processor circuit, the moment arm exceeds the moment arm threshold at any point during the fundamental period.

In some embodiments, the classifying the user's arm swing as a swimming motion may also include, extracting, by the processor circuit, a first set of features from the motion data. Embodiments may also include comparing, by the processor circuit, the first set of features to plurality of swimming motion features included in a motion classification model. Embodiments may also include matching, by the processor circuit, the first set of features with one or more features included in the plurality of swimming motion features.

In some embodiments, first set of features includes a period of time required to complete a stroke, one or more wrist poses of the user, and one or more motion features extracted from the rotational data. Embodiments may also include in response to detecting the start of the swimming activity, calculating, by the processor circuit, performance information of the user during the swimming activity, the performance information including a level of exertion based on a heart rate of the user measured by a heart rate sensor and the one or more swimming metrics.

In some embodiments, the one or more swimming metrics include turns, breaths, laps, swimming styles, and swimming strokes. Embodiments may also include in response to detecting the start of the swimming activity, outputting, by the processor circuit, the one or more swimming metrics on a display of the wearable device. In some embodiments, the one or more motion sensors may include at least one of an accelerometer and a gyroscope.

Embodiments may also include classifying, by the processor circuit, a swim stroke type based on the motion data and the rotational data. In some embodiments, the swim stroke type is at least one of a freestyle stroke, a breaststroke, a butterfly stroke, and a backstroke. In some embodiments, the classifying a swim stroke type based on the motion data and rotational data may include, extracting, by the processor circuit, a second set of features from the rotational data. Embodiments may also include matching, by the processor circuit, the second set of features with one or more features included in a swimming stroke motion profile.

In some embodiments, the second set of features include an orientation of the wearable device, a device angle, a range of motion feature, a moment arm length a correlation of the user's arm and wrist rotation, mean crown orientation during the fastest part of the stroke, a ratio of acceleration long two or more axis of rotation, a minimum rotation relative to a frame of reference, and a maximum rotation relative to a frame of reference.

In one aspect, disclosed herein are systems for improving performance of a wearable device while recording a swimming activity, the systems including one or more motion sensors configured to collect motion data of a user. Embodiments may also include a pressure sensor configured to collect pressure data. Embodiments may also include a processor circuit in communication with the one or more motion sensors and the pressure sensor, the processor circuit configured to execute instructions causing the processor circuit to, determine rotational data expressed in a frame of reference based on the motion data. In some embodiments, the processor circuit may also include detect a repeating pattern of user motion in the motion data. In some embodiments, the processor circuit may also classify a user's arm swing included in the repeating pattern of user motion as a swim stroke motion based on the motion data and the rotational data. In some embodiments, the processor circuit may also detect the swim stroke motion in the motion data for a first predetermined period of time. In some embodiments, the processor circuit may also confirm the user is swimming based on pressure data.

In some embodiments, the processor circuit is further configured to, sample a plurality of pressure signals from the pressure data. In some embodiments, the processor circuit may also continuously compare the plurality of pressure signals to a high pressure threshold. In some embodiments, the processor circuit may also detect at least one pressure signal that exceeds the high pressure threshold. In some embodiments, the processor circuit may also confirm the user is swimming in response to detecting the at least one pressure signal that exceeds the high pressure threshold.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1 is a diagram of an exemplary wearable device, according to embodiments of the disclosure.

FIG. 2 is a block diagram showing exemplary components that may be found within a wearable device, according to embodiments of the disclosure.

FIG. 3 is a flow chart illustrating a method for classifying motion, according to embodiments of the disclosure.

FIG. 4 is a flow chart illustrating a method for determining if a wearable device is submerged in water, according to embodiments of the disclosure.

FIGS. 5A-D illustrate methods for measuring the orientation of wearable devices relative to a fixed body frame of reference, according to embodiments of the disclosure.

FIG. 6 illustrates an inertial frame of reference, according to embodiments of the disclosure.

FIGS. 7A-D illustrate methods for measuring the orientation of wearable devices relative to an inertial frame of reference, according to embodiments of the disclosure.

FIG. 8 is a flow chart illustrating a method for detecting a swimming stroke motion, according to embodiments of the disclosure.

FIG. 9 is a graph illustrating the sensitivity of exemplary motion detection models for a variety of stroke motions, according to embodiments of the disclosure.

FIG. 10 is illustrates three exemplary stroke motions that may be performed while wearing a wearable device, according to embodiments of the disclosure.

FIG. 11 illustrates an example moment arm length, according to embodiments of the disclosure.

FIG. 12 is a graph illustrating motion data of a wearable device in a body-fixed frame of reference, according to embodiments of the disclosure.

FIG. 13 is a flow chart illustrating a method for confirming a swimming motion using pressure data, according to embodiments of the disclosure.

FIG. 14A is illustrates exemplary rotational data used to determine a user heading, according to embodiments of the disclosure.

FIG. 14B is graph including exemplary pressure data generated by a wearable device, according to embodiments of the disclosure.

FIG. 15 is a flow chart illustrating a method for classifying a swimming stroke, according to embodiments of the disclosure.

FIGS. 16A-B are graphs including exemplary moment arm calculations, according to embodiments of the disclosure.

FIGS. 16C-E display graphs including orientation data for classifying a user's swim stroke, according to embodiments of the disclosure.

FIG. 17 is a flow chart illustrated a method for determining the end of a swimming activity, according to embodiments of the disclosure.

### DESCRIPTION

The present disclosure describes systems and methods of detecting swimming activities using a wearable device. To accurately track a user's performance during a swimming workout it is important to quickly identify the beginning and end of swimming activities and the transitions between swimming stroke types. In various embodiments, the wearable device may track user performance during a swimming workout by detecting a swimming stroke by matching motion data with a swimming stroke profile included in a plurality of stroke profiles (e.g., a rowing stroke profile, a walking stroke profile, elliptical stroke profile, and the like). Swimming determinations made by the wearable device may be improved by confirming the start of a swimming activity using pressure data. Once a swimming activity is detected, the wearable device may classify the type(s) of swimming strokes performed during the activity to accurately track user performance during swimming activities.

Wearable devices may be used by users to track a variety of different activities. For users that are active for many hours of the day, it may be difficult to fully track each activity without recharging the wearable device and/or consuming a vast amount of network data and compute resources. Certain components of the device's battery, such as the main processor, Global Positioning System (GPS) receiver, and cellular module, all can draw a particularly high amount of power and consume a vast amount of network data and compute resources (e.g., memory, processing capacity, network communications, and the like). To minimize the amount of power, network data, and compute resources consumed by the wearable device, the systems and methods disclosed herein can detect when the user begins a swimming activity, ends a swimming activity, is stationary, begins performing a non-swimming activity, and the like. In response to detecting the end of a swimming activity, a stationary user, and/or performance of a non-swimming activity, the wearable device may transition from a tracking state to a low power state. One or more components of the wearable device may be selectively powered down certain when the device is in a low power state to increase battery life and reduce the amount of data and compute resources consumed. By minimizing the amount of time the wearable device is in a tracking state, the activity detection systems and methods disclosed herein can improve the functioning of wearable device by making them run longer on a single charge and more efficiently by consuming less data and compute resources to deliver the same functionality.

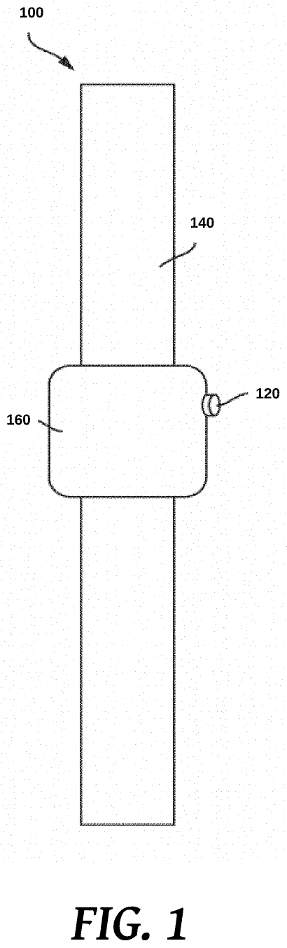

FIG. 1 shows an example of a wearable device 100 that may be worn by a user, in accordance with an embodiment of the present disclosure. In various embodiments, the wearable device 100 may be configured to be worn around the user's wrist using a band 140 (e.g., a watch strap). The wearable device may also have a crown 120 to orient the device and receive input from a user. The crown 120 may be positioned to the side of a display surface 160.

As described in more detail below, the wearable device 100 may be configured to detect the user's swimming activity, calculate performance information of the user during the swimming activity, detect the type of swimming stroke performed by the user, detect transitions between two or more different swimming strokes during a swimming workout, and provide additional functionality related to swimming activities to the user. In particular, the wearable device 100 may use motion data obtained from motion sensors, heart rate data obtained from a heart rate sensing module, orientation data obtained from a magnetic field sensor and/or motion sensors, and/or pressure data obtained from a pressure sensor to detect when the user begins a swimming activity, stops a swimming activity, transitions between two or more swimming strokes, classify a stroke motion as a swimming stroke, temporarily stops a swimming activity, performs a non-swimming activity and/or performs other swimming related activities. The wearable device may use a variety of motion data and orientation data to estimate the device direction which may be used to determine an angle feature and/or motion feature of a stroke performed by a user. Motion data and orientation data may be used by the wearable device to classify swimming motions and/or swimming stroke types performed by the user during a swimming workout.

To confirm a user is performing a swimming activity, pressure data may be used to detect when the wearable device is under water. Swimming metrics (e.g., speed distance, stroke type, swimming skill, and the like), heart rate, and user characteristics (e.g., age, maximum oxygen consumption, level of fitness, previous performance information, etc.) may be used by the wearable device to determine a user exertion level and/or a change in user exertion. Motion data, rotational data, and or direction information (e.g., user heading) may be used to confirm when a user starts and/or stops swimming.

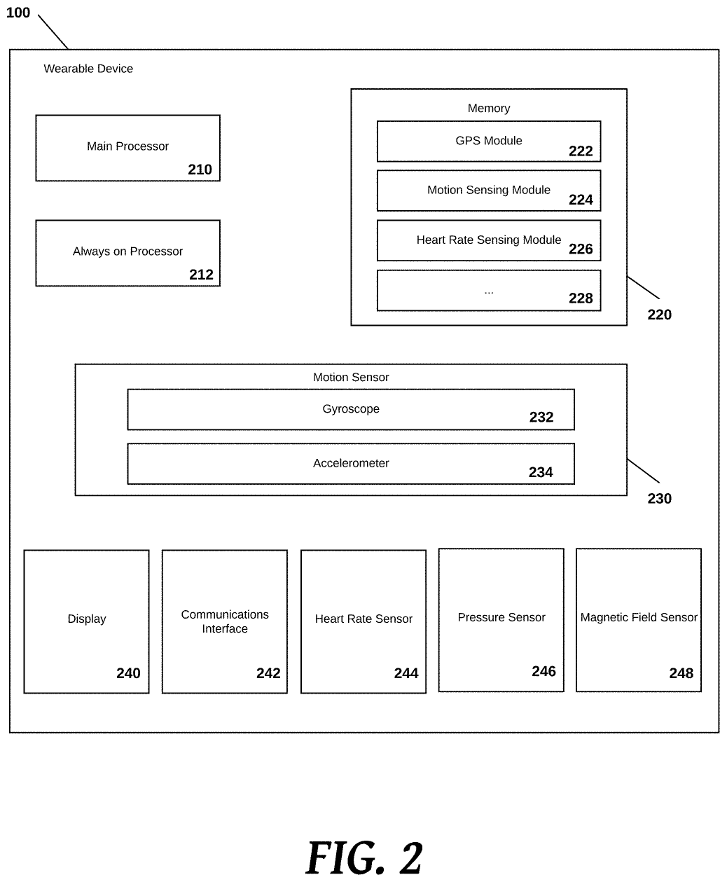

FIG. 2 depicts a block diagram of exemplary components that may be found within the wearable device 100 according to some embodiments of the present disclosure. In some embodiments, the wearable device 100 can include a main processor 210 (or “application processor” or “AP”), an always on processor 212 (or “AOP” or “motion co-processor”), a memory 220, one or more motion sensors 230, a display 240, an interface 242, a heart rate sensor 244, and a pressure sensor 246, and a magnetic field sensor 248. The wearable device 100 may include additional modules, fewer modules, or any other suitable combination of modules that perform any suitable operation or combination of operations.

In some embodiments, main processor 210 can include one or more cores and can accommodate one or more threads to run various applications and modules. Software can run on main processor 210 capable of executing computer instructions or computer code. The main processor 210 can also be implemented in hardware using an application specific integrated circuit (ASIC), programmable logic array (PLA), field programmable gate array (FPGA), or any other integrated circuit.

In some embodiments, wearable device 100 can also include an always on processor 212 which may draw less power than the main processor 210. Whereas the main processor 210 may be configured for general purpose computations and communications, the always on processor 212 may be configured to perform a relatively limited set of tasks, such as receiving and processing data from motion sensor 230, heart rate sensor 244, pressure sensor 246, and other modules within the wearable device 100. In many embodiments, the main processor 210 may be powered down at certain times to conserve battery charge, while the always on processor 212 remains powered on. Always on processor 212 may control when the main processor 210 is powered on or off.

Memory 220 can be a non-transitory computer readable medium, flash memory, a magnetic disk drive, an optical drive, a programmable read-only memory (PROM), a read-only memory (ROM), or any other memory or combination of memories. Memory 220 can include one or more modules 222-228.

The main processor 210 and/or always on processor 212 can be configured to run one or more modules 222-228 stored in memory 220 that are configured to cause main processor 210 or always on processor 212 to perform various steps that are discussed throughout the present disclosure.

In some embodiments, the wearable device 100 can include one or more motion sensors 230. For example, motion sensors 230 can include a gyroscope 232 and an accelerometer 234. In some embodiments, accelerometer 234 may be a three-axis accelerometer that measures linear acceleration in up to three-dimensions (for example, x-axis, y-axis, and z-axis). In some embodiments, gyroscope 232 may be a three-axis gyroscope that measures rotational data, such as rotational movement and/or angular velocity, in up to three-dimensions (for example, yaw, pitch, and roll). In some embodiments, accelerometer 234 may be a microelectromechanical system (MEMS) accelerometer, and gyroscope 232 may be an MEMS gyroscope. Main processor 210 or always on processor 212 of wearable device 100 may receive motion information from one or more motion sensors 230 to track acceleration, rotation, position, or orientation information of wearable device 100 in six degrees of freedom through three-dimensional space.

In some embodiments, the wearable device 100 may include other types of sensors in addition to accelerometer 234 and gyroscope 232. For example, the wearable device 100 may include a pressure sensor 246 (e.g., an altimeter, barometer, and the like), a magnetic field sensor 248 (e.g., a magnetometer, compass, and the like) and/or a location sensor (e.g., a Global Positioning System (GPS) sensor). The pressure sensor may be able to detect pressure up to 110 kilopascals (kPa).

The wearable device 100 may also include a display 240. The display 240 may be a screen, such as a crystalline (e.g., sapphire) or glass touchscreen, configured to provide output to the user as well as receive input from the user via touch. For example, the display 240 may be configured to display a current heart rate or daily average energy expenditure. The display 240 may receive input from the user to select, for example, which information should be displayed, or whether the user is beginning a physical activity (e.g., starting a session) or ending a physical activity (e.g., ending a session), such as a cardio machine session, a swimming session, a running session, or a cycling session. In some embodiments, wearable device 100 may present output to the user in other ways, such as by producing sound with a speaker, and wearable device 100 may receive input from the user in other ways, such as by receiving voice commands via a microphone.

In various embodiments, wearable device 100 may communicate with external devices via an interface 242, including a configuration to present output to a user or receive input from a user. The interface 242 may be a wireless interface. The wireless interface may be a standard Bluetooth® (IEEE 802.15) interface, such as Bluetooth® v4.0, also known as “Bluetooth low energy.” In various embodiments, the interface may operate according to a cellphone network protocol such as Long Term Evolution (LTE™) or a Wi-Fi (IEEE 802.11) protocol. In various embodiments, the interface 242 may include wired interfaces, such as a headphone jack or bus connector (e.g., Lightning®, Thunderbolt™, USB, etc.).

In some embodiments, wearable device 100 can measure an individual's current heart rate from a heart rate sensor 244. The heart rate sensor 244 may also be configured to determine a confidence level indicating a relative likelihood of an accuracy of a given heart rate measurement. In various embodiments, a traditional heart rate monitor may be used and may communicate with wearable device 100 through a near field communication method (e.g., Bluetooth).

In various embodiments, the wearable device 100 can include a photoplethysmogram (PPG) sensor. PPG is a technique for measuring a person's heart rate by optically measuring changes in the person's blood flow at a specific location. PPG can be implemented in many different types of devices in various forms and shapes. For example, a PPG sensor can be implemented in a wearable device 100 in the form of a wrist strap, which a user can wear around the wrist. A PPG sensor may also be implemented on the underside of a wearable device 100. The PPG sensor can optically measure the blood flow at the wrist. Based on the blood flow information, the wearable device 100 can derive the person's heart rate.

The wearable device 100 may be configured to communicate with a companion device, such as a smartphone. In various embodiments, wearable device 100 may be configured to communicate with other external devices, such as a notebook or desktop computer, tablet, headphones, Bluetooth headset, etc.

The modules described above are examples, and embodiments of wearable device 100 may include other modules not shown. For example, some embodiments of wearable device 100 may include a rechargeable battery (e.g., a lithium-ion battery), a microphone array, one or more cameras, two or more speakers, a watchband, water-resistant casing or coating, etc. In some embodiments, all modules within wearable device 100 can be electrically and/or mechanically coupled together. In some embodiments, main processor 210 and or always on processor 212 can coordinate the communication among each module.

In various embodiments, the wearable device 100 may use sensed and collected motion information (e.g., acceleration data, rotational data, directional data, and the like) to predict a user's activity. Examples of activities may include, but are not limited to cardio machine activities, walking, running, cycling, swimming, skiing, etc. Wearable device 100 may also be able to predict or otherwise detect when a user is stationary (e.g., sleeping, sitting, standing still, driving, etc.). Wearable device 100 may use a variety of motion data, device orientation data, directional information and/or pressure data to predict a user's activity.

Wearable device 100 may use a variety of heuristics, algorithms, or other techniques to predict the user's activity and/or detect activity start and end points. In various embodiments, one or more machine learning techniques and/or predictive models trained on a plurality of datasets may be used to predict the user's activity and/or detect activity start and end points. The plurality of datasets may include motion data, device orientation data, directional information, pressure data, and the like measured during a plurality of swimming activities and or other activity types. The activities included in the plurality of datasets may be performed by the user and or a group of users. The group of users may have one or more characteristics (e.g., age, fitness level, cardio vascular heath, swimming skill level, and the like) in common with the user. Wearable device 100 may also estimate a confidence level (e.g., percentage likelihood, degree of accuracy, etc.) associated with a particular prediction (e.g., 90% likelihood that the user is cycling) or predictions (e.g., 60% likelihood that the user is cycling and 40% likelihood that the user is performing some other activity).

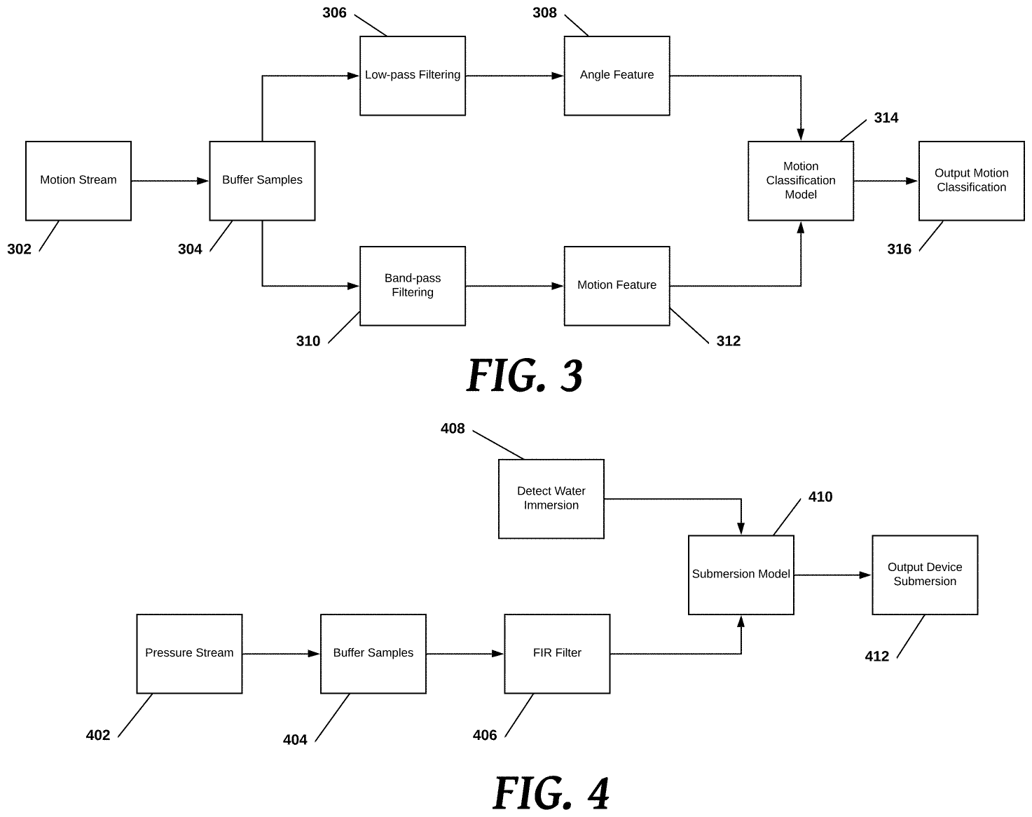

FIG. 3 illustrates an exemplary motion classification method of the present disclosure. At block 302, a stream of motion data (e.g., accelerometer data, gyroscope data, orientation data, and the like) may be provided to block 304. At block 304, samples of motion data from the stream of motion data may be taken at a suitable resolution (e.g., 200 samples). 200 samples may be taken at a frequency of 100 Hz over a time period of 2 seconds. In other embodiments, other sample rates may be used (e.g., 256 samples or 1,000 samples), or other sample frequencies may be used (e.g., 50 Hz, or 200 Hz) to buffer the samples. Copies of the samples may be passed to blocks 306 and 310. In various embodiments, longer duration epochs, for example 10 second epochs may be taken to collect 1,000 or more samples. However, samples from epochs having longer durations may provide less accurate motion classification because multiple motion postures could occur within a single epoch (e.g., a longer ten-second epoch). Similarly, motion classifications made using shorter epochs may be less accurate because they may not provide a sufficient number of samples to detect a motion posture accurately. The most accurate epoch duration to use for motion classification may be determined from a plurality of datasets including motion data captured during swimming activities and or other activity types. At block 306, low-pass filtering (LPF) may be performed to subtract out frequencies attributable to fidgeting or other incidental motion. The remaining information (i.e., the portion of motion data that tends to change slowly) may reflect the orientation of the wearable device 100 (e.g., the angle of the wearable device 100 with respect to the horizon, explained in detail below). For example, if a user is standing with his arms at the user's side, the user might be fidgeting, but the angle of the fitness tracking device on the user's arm (e.g., approximately −π/2 radians) may only vary slightly during a short time period (e.g., 2-3 seconds). This angle information may be represented in relatively low frequencies (e.g., less than 0.5 Hz, or less than 1.0 Hz), and this low frequency signature may be passed to block 308.

At block 308, the relatively low frequency signature from block 306 may be used to compute the angle of the wearable device 100 with respect to a horizon plane (e.g., the X-Y plane parallel to the ground). The computed angle (e.g., approximately −π/2 radians) may be passed to block 314 as one of the inputs into the motion classification model at block 314.

At block 310, band-pass filtering may be performed to obtain human motion information contained in a relatively higher frequency band (e.g., over 0.5 Hz, or over 1.0 Hz, and up to 5.0 Hz, or 4.0 Hz), such as fidgeting or other incidental motion. This band's frequency signature may be passed to block 312. The band-pass filter may be configured with a frequency band (e.g., 1.0-5.0 Hz) tuned to capture human motion likely to occur when the user is sedentary (e.g., fidgeting or incidental motion). In some embodiments, if it may be determined that more rapid (e.g., higher frequency) human motion is likely to occur while sedentary, the frequency band of the band-pass filter may be calibrated or otherwise adjusted accordingly.

At block 312, the band's frequency signature from block 310 may be used to predict a motion posture based on the motion data received from block 310. In some embodiments, the prediction may be based on the assumption that the range of motion (e.g., range of wrist motion, such as when a user's arms are swaying) while standing is likely to be greater than the range of motion (e.g., wrist motion) while the user is sitting. The range of motion may be estimated based on the motion data received from block 310, which may have been filtered using a band-pass filter to include motion that may likely be attributable to human motion (e.g., fidgeting).

In some embodiments, the relative range of motion during a given time period (or “epoch”) may be represented as a range of amplitudes of accelerometer values. For example, the interquartile range (IQR) between the 75th percentile and 25th percentile accelerometer amplitudes over a number of samples (e.g., 250 samples) during the time period may be considered for the range of motion. For example, it some embodiments, a typical separation in ranges of motions for IQR while sitting as opposed to IQR while standing may be determined to be greater than or equal to approximately 0.1 to 0.2 meters.

Furthermore, it may be determined that a particular axis or combination of axes of a three-axis accelerometer within the wearable device 100 provides the most reliable IQR to distinguish between motion likely occurring while standing as opposed to motion likely occurring while sitting. In some embodiments, for example, the x-axis of the accelerometer may be determined to provide the most reliable IQR. In other embodiments, a default axis or weighted combination of axes may be selected, and the selected axis or axes may be calibrated or otherwise adjusted based on individual use. For example, the typical incidental motion for a user relative to the typical position and orientation of the wearable device 100 on the user's wrist or other part of the body may affect which axis or combination of axes may provide the most useful range of motion data for the IQR motion feature. This IQR motion feature may be passed to block 314 as a second input into the motion classification model at block 314.

At block 314, the computed angle feature and the computed IQR motion feature may be considered in the model classification model (e.g., a decision tree including a sequence of “if-else” conditional branches using a model with thresholds for angle and motion values, a machine learning model that becomes more accurate over time by training on a plurality of datasets including motion data, and the like). Once a motion classification has been made (e.g., the user is swimming, the user is running, the user is performing a stroke motion, the user is stationary and sitting, the user is stationary and standing, and the like), the classification may be output at block 316. In various embodiments, the motion classification may be a decision tree, for example:

If (Angle < −0.5 radians and IQR > 0.1)
Return “Standing”
Else If (−0.5 radians < Angle < 0 radians and IQR > 0.2)
Return “Standing”
Else If . . .

In the example decision tree above, the first condition (Angle<−0.5 radians and IQR>0.1) may represent typical parameters when a user's hands are well below a horizon, though not necessarily vertically at the user's sides. Because the angle may be sufficiently unambiguous, it may be less important to observe a relatively large IQR to estimate that the user is probably standing. In the second condition (−0.5 radians<Angle<0 radians and IQR>0.2), the angle may be considered more shallow, and more ambiguous (e.g., the user's arms are crossed). In this situation, a relatively ambiguous angle may make it relatively more important to observe a relatively large IQR to estimate that the user is probably standing.

Other conditions, or additional portions of the conditions listed above, may be included in other embodiments. For example, another condition (not shown above) may indicate that if the angle is a positive value, it may be predicted that the user is likely sitting. Alternatively, some positive angles may be considered ambiguous. Another feature for resolving ambiguous angle and IQR features may include a pedometry feature. For example, if a pedometer function of the wearable device 100 may confirm that the user may be walking, running, striding on an elliptical machine, or performing another step based motion.

In some embodiments, another feature to consider may be the frequency or rapidity of incidental movement (as opposed to the IQR feature described above, which indicates the amplitude or range of incidental movement). Frequency of movement may be determined by observing the number of zero crossings (or variations around the mean) of a value of one or more axes of an accelerometer.

In some embodiments, other features in addition to, or instead of, angle and IQR motion may be considered to classify motion, such as differences between X, Y, and Z accelerometer channels; mean values, vector magnitude, activity counts (e.g., how many times a signal crosses a stepping/stationary threshold in the epoch window), spectral power, etc. may be considered when classifying the user's motion. In various embodiments, it may be determined that angle and IQR may be less effective than other features for detecting whether a user is performing a particular motion. For example, motions performed with restricted wrist/arm movement (e.g., swimming without preforming an arm stroke, floating, and/or gliding under water) may have little to no change in angle and IQR during the motion relative to motions performed without restricted wrist/arm movement (e.g., performing a freestyle swimming stroke, a butterfly swimming stroke, and/or other swimming motion including an arm stroke). Therefore, the wearable device may classify motion using features other than IQR and angle to classify motions with restricted arm/wrist motion.

To classify motions with restricted arm/wrist motions, an activity count may be more likely to predict whether a user is performing an activity or stationary. In some embodiments, the counted activity may be zero crossings over the angle threshold (e.g., angle crossings over the horizon). For example, it may be determined that the angle feature is more likely to cross a threshold angle more frequently when the user is swimming as opposed to when the user is stationary or sitting, in which case a higher activity count (measured by threshold angle crossings) may be a more accurate predictor of motion in this situation.

In some embodiments, other classifiers in addition to, or instead of, the decision tree may be used to classify motion based on the one or more input features (e.g., the angle and IQR motion features). For example, random forests, a separate sit detector, a separate stand detector, support vector machines, etc., may be used to classify or otherwise classify the user's motion.

In some embodiments, a feedback or hysteresis mechanism may be used to smooth out possible noise in the detection output. For example, the method may track the previous four epoch states (or more or fewer epoch states) and consider a confidence level or other indicator of which of the current or prior epoch states may be determined to be the dominant or most confident indicator of posture.

In some embodiments, the classifier (e.g., the motion classification model used at block 316) may be biased toward detecting a stationary posture more frequently. For example, ambiguous states may be more likely to resolved as a stationary posture instead of an activity posture (e.g., walking, running, swimming, cycling, and the like). In this situation, there may be fewer false positives for an activity posture, which makes it less likely that users who are stationary will not receive additional credit for extra energy expenditure for an activity while they were stationary. In other embodiments, the decision tree may be biased to break-ties in favor of activity motions, which may make it less likely that a user who is performing an activity may be docked credit for a false positive stationary detection.

FIG. 4 illustrates an exemplary method for detecting water submersion of the wearable device using pressure data obtained from a pressure sensor. Block 402, may receive a stream of pressure data (e.g., pressure stream) from the pressure sensor of the wearable device. The raw pressure signal may be provided to block 404. At block 404, samples of pressure data from the stream of pressure data may be taken at a suitable resolution (e.g., 200 samples). 200 samples may be taken at a frequency of 100 Hz over a time period of 2 seconds. In other embodiments, other sample rates may be used (e.g., 256 samples or 1,000 samples), or other sample frequencies may be used (e.g., 50 Hz, or 200 Hz). Copies of the samples may be passed to block 406. In various embodiments, a longer duration epoch (e.g., 10 or more seconds) may be taken to collect 1,000 or more samples. However, longer durations may provide less accurate submersion predictions because multiple submerged and unsubmerged states could occur during a series of swimming strokes performed within a single epoch (e.g., a longer ten-second epoch). Similarly, submersion predictions made using shorter epochs may be less accurate because they may not provide a sufficient number of samples to detect a submerged or unsubmerged state accurately. The most accurate epoch duration to use for detecting submersion may be determined from a plurality of datasets including pressure data captured during swimming activities and or other activity types.

At block 406, the pressure samples can be filtered to improve data quality. Pressure data, in particular, can be noisy making it difficult to extract reliable, accurate information from the raw data. In various embodiments, a filter (e.g., a finite impulse response (FIR) filter) may be used to smooth the raw pressure data in order to get a more accurate estimate of device submersion. In response to detecting water immersion (e.g., water on the surface of the device) at block 408, filtered pressure data may be provided to block 410. In various embodiments, a water immersion model may detect an amount of water on the wearable device using data from one or more sensors (e.g., a pressure sensor, electrical sensor, optical sensor, and the like). In various embodiments, the water immersion model may use pressure data from a pressure sensor to detect weight on the surface of the device. The water immersion model may use electrical signal(s) measured by one or more electrical sensors (e.g., electrodes) to detect an amount of water on the wearable device by determining an increase and/or decrease in electrical resistance. The water immersion model may also use optical data from an optical sensor (e.g., camera) to detect an amount of water of the wearable device by detecting a decrease in photo resolution.

At block 410, epochs (e.g., time periods) of filtered pressure data may be analyzed to determine if a wearable device having a detected water immersion event is submerged in water using a submersion model (e.g., a decision tree including a sequence of “if-else” conditional branches using a model with thresholds for pressure values, a machine learning model that becomes more accurate over time by training on a plurality of datasets including pressure data captured during swimming activities and other activity types, and the like). Once a submersion decision (e.g., device submerged, device not submerged) has been made, the classification may be output at block 412. In various embodiments, the motion classification may be a decision tree, for example:

If (Immersion = Yes and Pressure > 500 Pa)
Return “Submerged”
Else If (Immersion = Yes and 5 Pa < Pressure < 500 kPa)
Return “Not Submerged”
Else If . . .

In the example decision tree above, the first condition (Immersion=Yes and Pressure>500 pascals (Pa)) may represent typical parameters when a wearable device is submerged in water while a user is swimming. In various embodiments, the immersion detector may determine the wearable device is in contact with water by detecting a water immersion. Water immersion be detected based on pressure data, electrical signal, and/or optical data. In various embodiments, to detect a water immersion, pressure data measured by the pressure sensor is compared to a water immersion threshold (e.g., 0.5-10 Pa of pressure). If the measured pressure exceeds the water immersion threshold, the wearable device may detect a water immersion. The pressure may then be compared to a water submersion threshold (e.g., >500 kPa) to determine if the device is submerged in water. The immersion detector may detect a submersion event when the wearable device is submerged in water and when the wearable device has an amount of water on a surface. Therefore, pressure data, specifically high pressure (e.g., more than about 500 Pa of pressure), may be used to detect device submersion. In the detected pressure is within the range of water immersion (i.e., 1<Pressure<500 kPa), but falls short of the water submersion threshold (i.e., >500 kPa) the submersion model will estimate that the device is not submerged despite detecting a water immersion event.

In some embodiments, other classifiers in addition to, or instead of, the decision tree may be used to detect device submersion based on the one or more input features (e.g., pressure data, optical data, electrical signal, and the like). For example, random forests, a separate sit detector, a separate stand detector, support vector machines, etc., may be used to detect or otherwise classify device submersion. The water immersion threshold and or the water submersion threshold may be determined from a plurality of datasets include pressure data captured during activities that include at least one water immersion and or water submersion events.

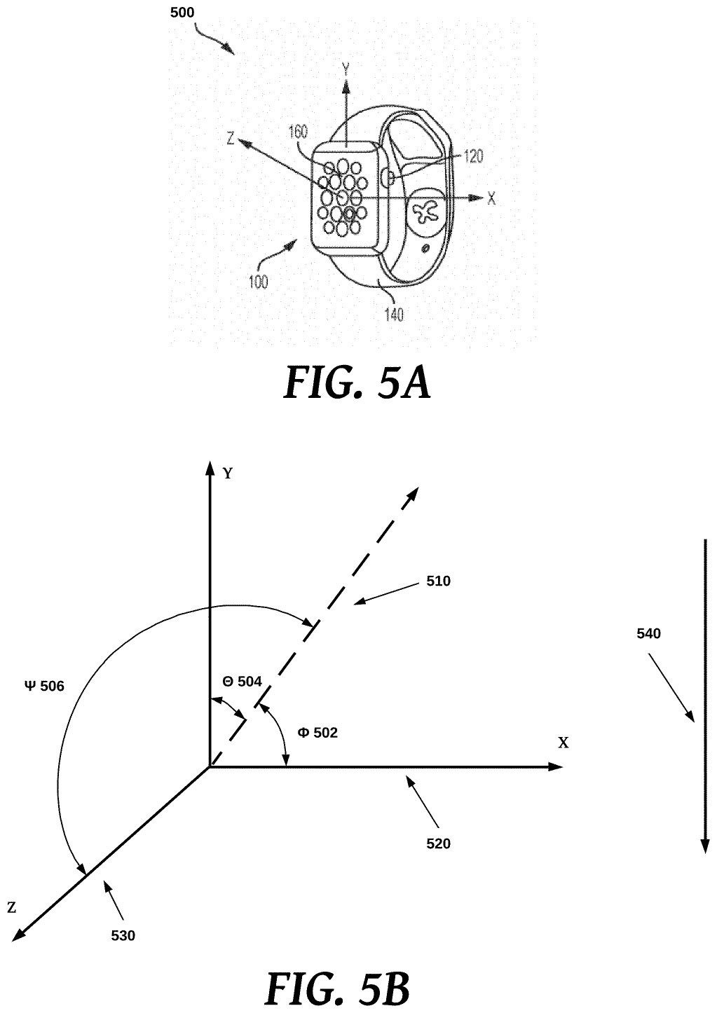

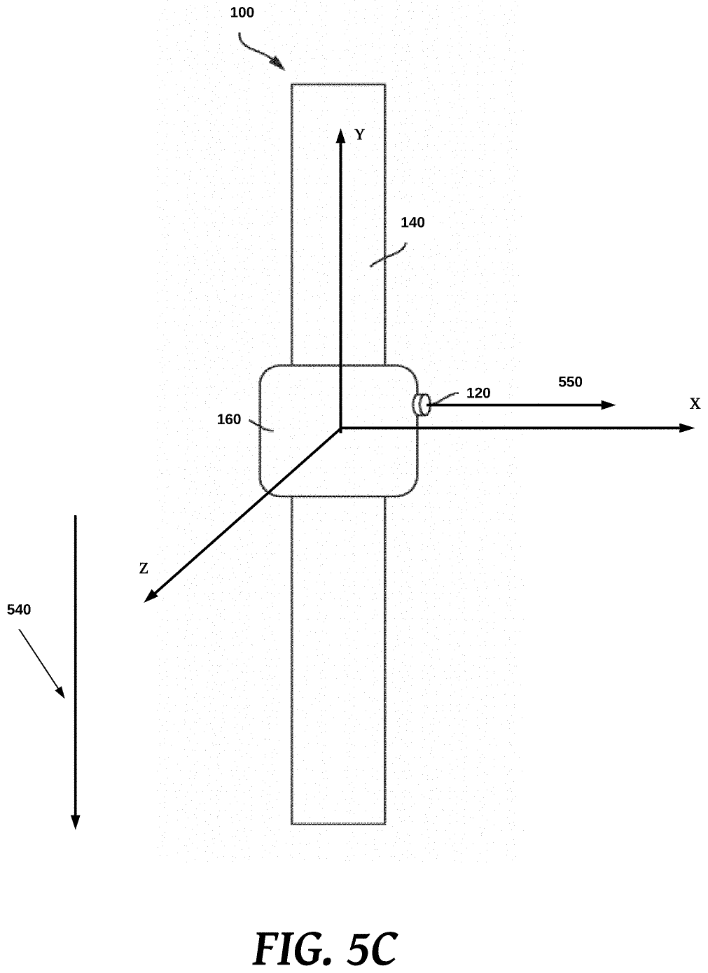

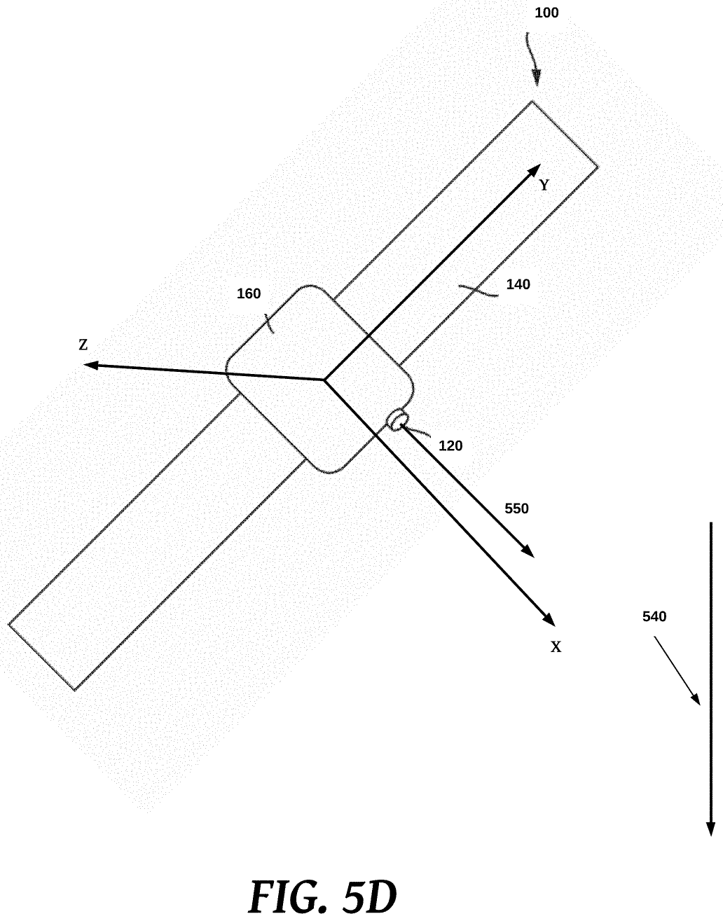

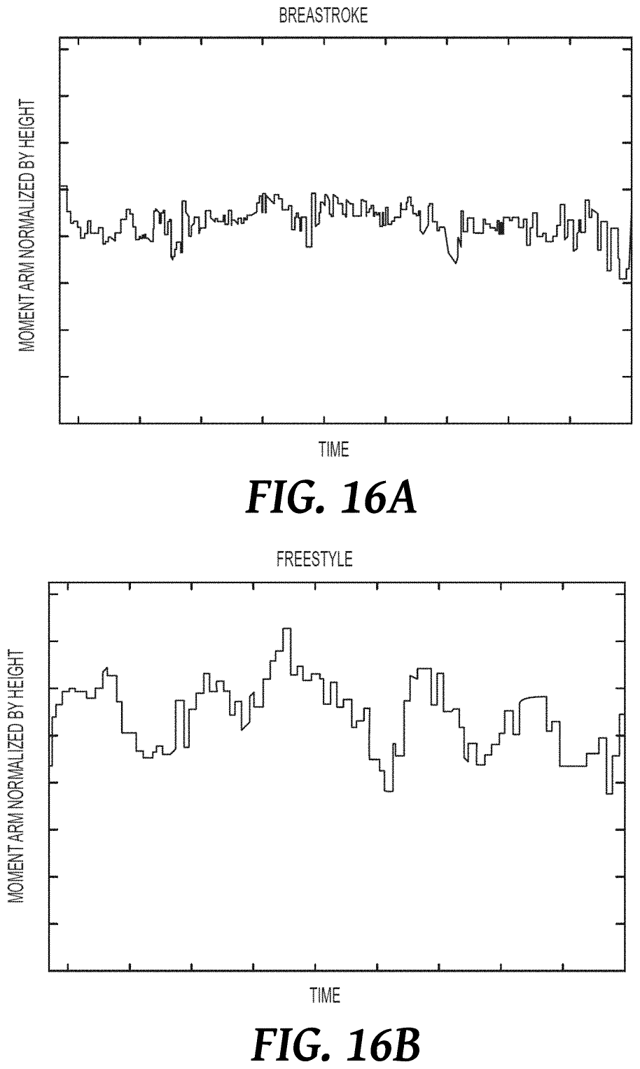

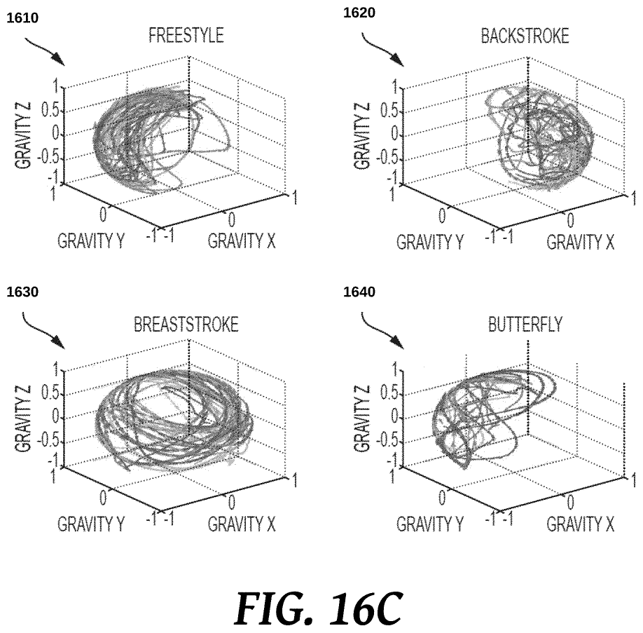

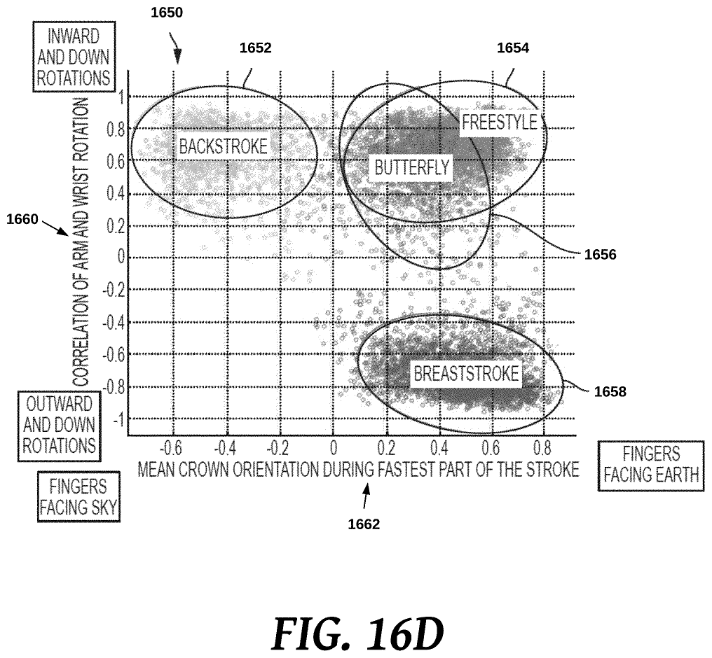

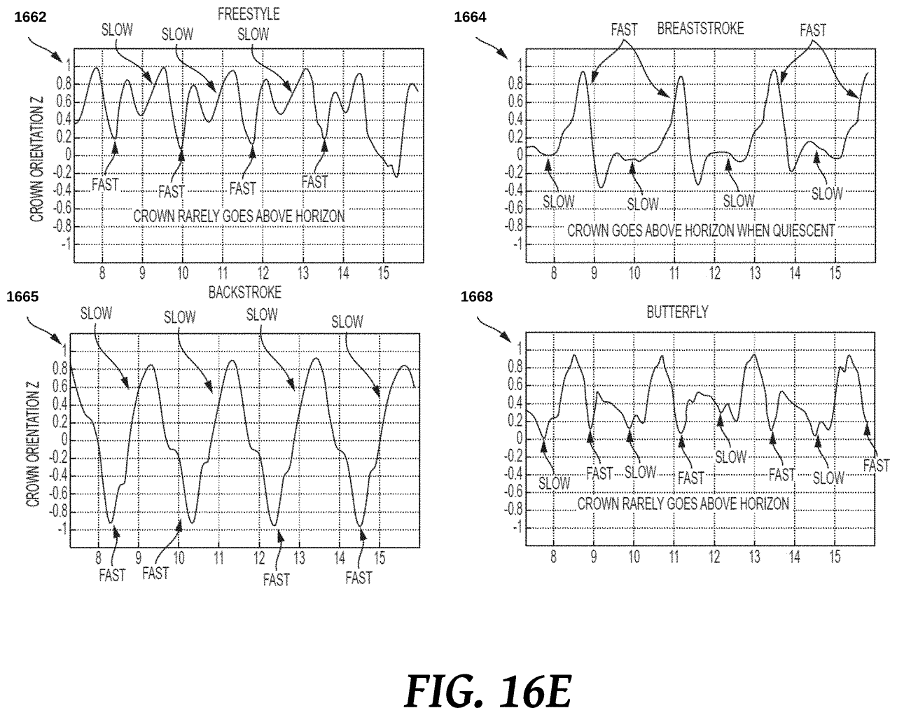

FIGS. 5A-7D describe exemplary methods for estimating rotational data of a wearable device according to the present disclosure. The rotational data may describe a device orientation at a particular point in time during a swimming workout or other activity. Device orientations may include the direction of the device in relation to one or more axes of rotation within a fixed body frame of reference (e.g., a frame of reference relative to the earth, the device, and the like). The device orientation/direction may be generated by applying one or more trigonometric functions (e.g., sine (sin), cosine (cos), tangent (tan), cosecant (csc), secant (sec), and cotangent (cot)) to one or more angles describing position relative to an axis of rotation (e.g., yaw, pitch, and roll) or other rotational data. As shown in FIG. 16C (discussed in detail below), device directions may be plotted in a 3D dimensional space bounded by three axes having a range of range of values between −1 and 1 to generate device orientation datasets that may be used to classify motion performed by the user (e.g., swim stroke types). A motion classification model may classify user motion by detecting one or more clusters, groups, sequences, patterns, and/or heuristics of the device orientations included in a plurality of datasets including rotational data and or device orientation data from swimming activities and or other activity types. For example, the motion classification model may determine a user is performing a swimming motion based on the motion detection model described above at FIG. 3.

Device orientation and or other rotational data may then be used to confirm the swimming motion. For example, the start of a swimming activity may be determined based on a user heading or change in user heading. Heading may describe the user's direction of travel and steady state changes in user heading detected from rotational data may be used to determine when a user performs a turn while swimming laps. In the 3D device orientation dataset described above, the number of clusters included in the orientation dataset may correspond to the number of distinct device directions of travel within a swimming activity. Accordingly, orientation datasets having rotational data contained within one cluster may correspond to swimming activities having zero direction of travel changes (i.e., a constant user heading) and no turns. Detecting a device orientation outside of the cluster, however, may indicate a change in the user's direction of travel change. The magnitude and frequency of the changes in the user's direction of travel detected by the wearable device may serve as primary indicators that a user is performing a specific swimming stroke type and or help confirm that the user is swimming.

In various embodiments, rotational data and device orientations may be generated according to techniques described in U.S. patent application Ser. No. 15/691,245, filed on Aug. 30, 2017, and entitled “SYSTEMS AND METHODS FOR DETERMINING SWIMMING METRICS,” which patent application is incorporated herein in its entirety.

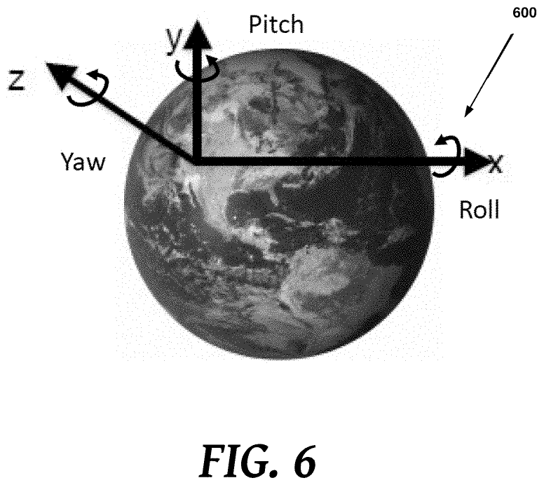

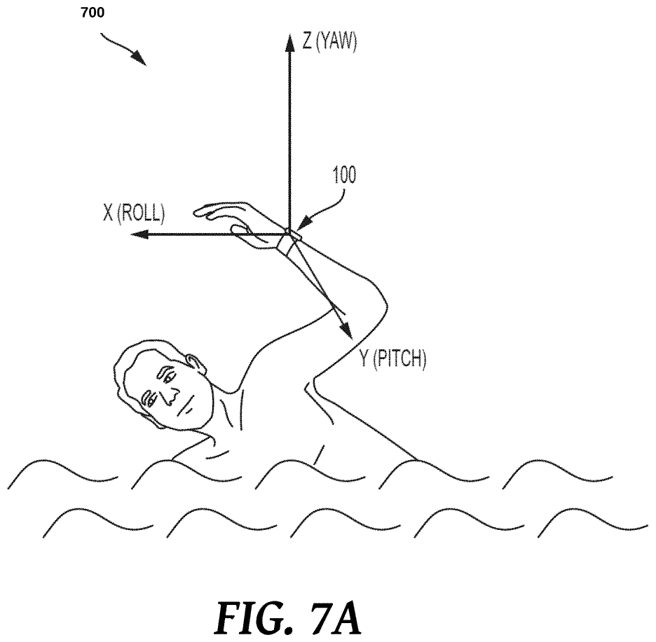

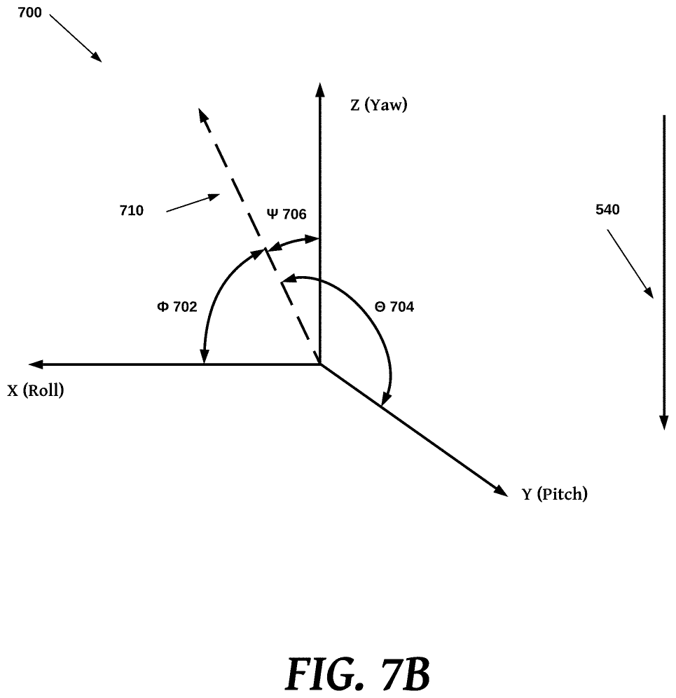

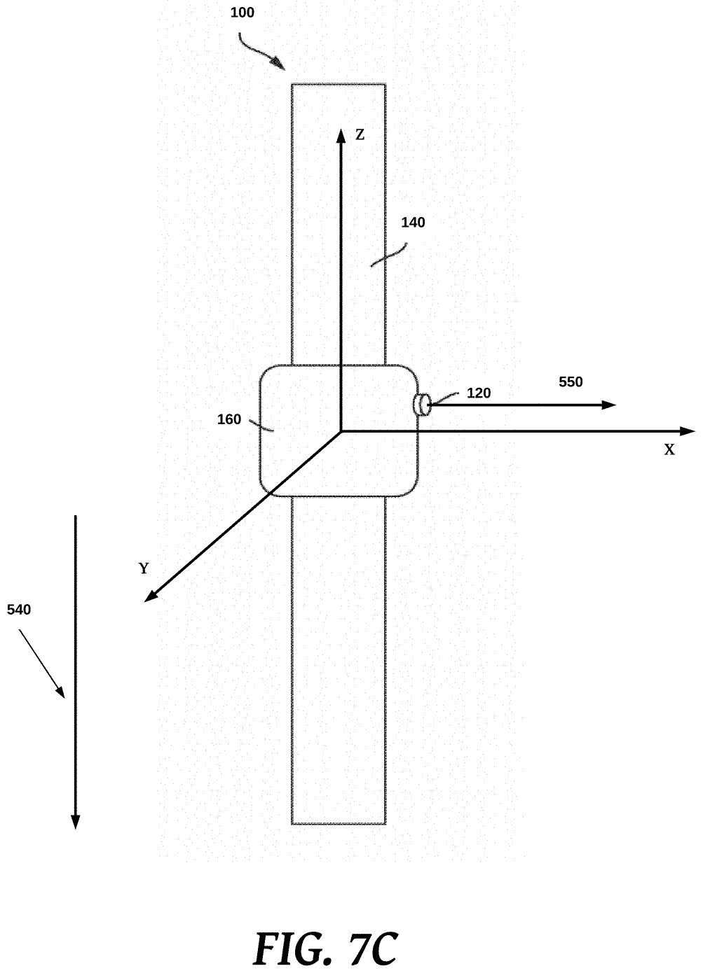

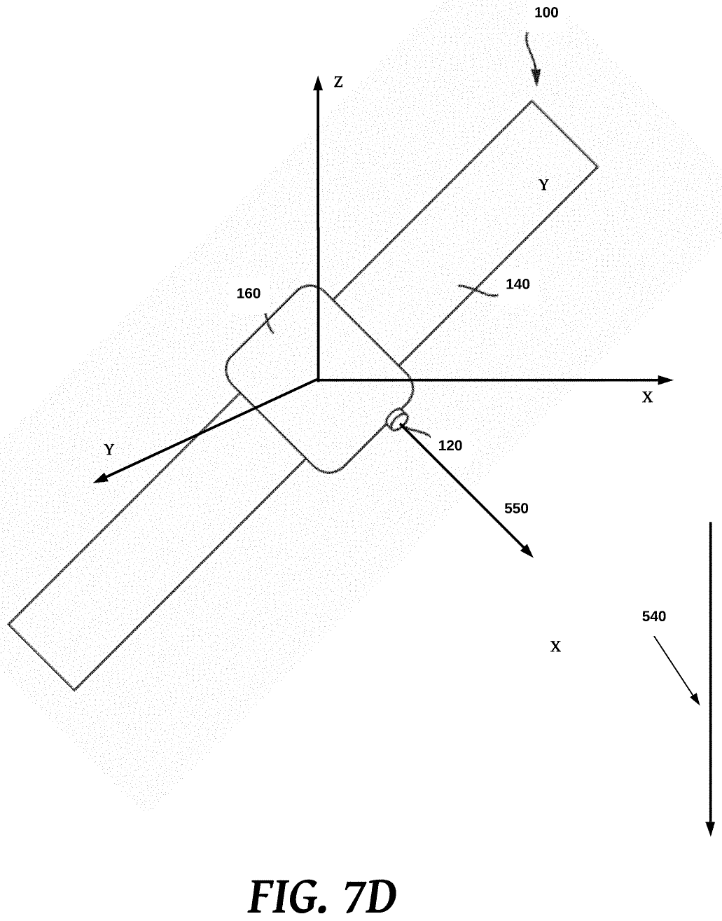

In various embodiments, rotational data may be used to determine the position of a wearable device relative to a frame of reference. FIGS. 5A-D describe rotational data generated relative to a body fixed frame of reference and FIGS. 6-7D describe rotational data generated relative to a inertial frame of references.

FIG. 5A illustrates an example of a body-fixed frame of reference 500 according to various embodiments of the present disclosure. In FIG. 5A, the rotational axes of body-fixed frame of reference 500 are with respect to wearable device 100. For example, the z-axis is perpendicular to the display surface 160 of wearable device 100. The x-axis and the y-axis can be chosen relatively arbitrarily as long as the three axes are perpendicular to each other. In FIG. 5A, the x-axis is parallel with the direction pointed by a crown 120 of wearable device 100, and the y-axis is parallel with the direction of the band 140 of wearable device 100 (assuming the direction pointed by the crown 120 of wearable device 100 is perpendicular to the direction of the band 140 of wearable device 100).

FIG. 5B-5D illustrate exemplary rotational data describing the orientation of the wearable device in body-fixed frame of reference 500. In FIG. 5B, the device orientation 510 has rotational data including an angle (ϕ) 502 with respect to the positive x-axis, an angle (θ) 504 with respect to the positive y-axis, and an angle (ψ) 506 with respect to the positive z-axis. The device orientation 510 can be expressed in body-fixed frame of reference 500 (i.e., relative to the body of the wearable device), for example, as [cos(ϕ), cos(θ), cos(ψ)]. FIG. 5B illustrates a second device orientation 520 that is parallel with and pointing toward the positive x-axis. The rotational data for the second device orientation 520 includes the angle (ϕ) between the second device orientation 520 and the positive x-axis measuring 0-degrees; the angle (θ) between the second device orientation 520 and the positive y-axis measuring 90-degrees; and the angle (ψ) between the second device orientation 520 and the positive z-axis measuring 90-degrees. Therefore, the second device orientation 520 can be expressed as [cos(0), cos(90), cos(90)], which is [1, 0, 0]. FIG. 3 further illustrates a third device orientation 530 that parallel with and pointing toward the positive z-axis. The third device orientation 530 has rotational data that includes the angle (ϕ) between the third device orientation 530 and the positive x-axis measuring 90-degrees; the angle (θ) between the third device orientation 530 and the positive y-axis measuring 90-degrees; and the angle (ψ) between the third device orientation 530 and the positive z-axis measuring 0-degrees. Therefore, the third device orientation 530 can be expressed as [cos(90), cos(90), cos(0)], which is [0, 0, 1]. As yet another example, a fourth device orientation 540 is shown in FIG. 5B. The fourth device orientation 540 represents the direction of gravity in FIG. 5B and is parallel with and pointing toward the negative y-axis. Accordingly, the rotational data for the fourth device orientation 540 includes the angle (ϕ) between the fourth device orientation 540 and the positive x-axis measuring 90-degrees; the angle (θ) between the fourth device orientation 540 and the positive y-axis measuring 180-degrees; and the angle (ψ) between the fourth device orientation 540 and the positive z-axis measuring 90-degrees. Therefore, the fourth device orientation 540 can be expressed as [cos(90), cos(180), cos(90)], which is [0, −1, 0].

In FIG. 5C, wearable device 100 is held vertically. As discussed earlier, in the fixed body frame of reference, the x-axis is parallel with direction pointed by the crown 120, the y-axis is parallel with the band 140, and the z-axis is perpendicular to the display surface 160. The fifth device orientation 550 shown in FIG. 5C is aligned with the direction pointed by the crown 120. Accordingly, the rotational data for the fifth device orientation includes the angle (ϕ) between the fifth device orientation 550 and the positive x-axis measuring 0-degrees; the angle (θ) between the fifth device orientation 550 and the positive y-axis measuring 90-degrees; and the angle (ψ) between the fifth device orientation 550 and the positive z-axis measuring 90-degrees. Therefore, the fifth device orientation 550 can be expressed as [cos(0), cos(90), cos(90)], which is [1, 0, 0]. As another example, fourth device orientation 540 represents direction of gravity in FIG. 5C and is parallel with and pointing toward the negative y-axis. Accordingly, the rotational data for of the fourth device orientation 540 includes the angle (ϕ) between fourth device orientation 540 and the positive x-axis measuring 90-degrees; the angle (θ) between the fourth device orientation 540 and the positive y-axis measuring 180-degrees; and the angle (ψ) between fourth device orientation 540 and the positive z-axis measuring 90-degrees. Therefore, fourth device orientation 540 in FIG. 5C can be expressed as [cos(90), cos(180), cos(90)], which is [0, −1, 0].

In FIG. 5D, wearable device 100 is rotated 45-degree clockwise compared with FIG. 5C. As discussed earlier, the x-axis is parallel with direction pointed by the crown 120, the y-axis is parallel with the band 140, and the z-axis is perpendicular to the display surface 160. The fifth device orientation 550 in FIG. 5D represents the direction pointed by the crown 120. Accordingly, the rotational data for the fifth device orientation 550 includes the angle (ϕ) between the fifth device orientation 550 and the positive x-axis measuring 0-degrees; the angle (θ) between the fifth device orientation 550 and the positive y-axis measuring 90-degrees; and the angle (ψ) between the fifth device orientation 550 and the positive z-axis measuring 90-degrees. Therefore, the fifth device orientation 550 can be expressed as [cos(0), cos(90), cos(90)], which is [1, 0, 0]. Similar to FIGS. 5B and 5C, the fourth device orientation 540 represents direction of gravity in FIG. 5D. The angle (ϕ) between the fourth device orientation 540 and the positive x-axis is 45-degrees; the angle (θ) between the fourth device orientation 540 and the positive y-axis is 135-degrees; and the angle (ψ) between the fourth device orientation 540 and the positive z-axis is 90-degrees. Therefore, the fourth device orientation 540 in FIG. 5D can be expressed as [cos(45), cos(135), cos(0)], which is [0.707, −0.707, 0].

It is noted that the fifth device orientation 550 is the same in FIG. 5C and FIG. 5D even though wearable device 100 has rotated. This is because the body-fixed frame of reference 500 is always fixed with respect to wearable device 100. As a result, when position of wearable device 100 changes, the three axes in body-fixed frame of reference 500 and fifth device orientation 550 change too, and relative position between the fifth device orientation 550 and the three axes remain the same. On the other hand, although direction of gravity does not change in an “absolute” sense, it does not rotate together with wearable device 100. Therefore, the expression of gravity's orientation that corresponds to the fourth device orientation 540 changes in the body-fixed frame of reference 500 when wearable device changes position.

FIG. 6 illustrates an inertial frame of reference 600 according to some embodiments of the present disclosure. In FIG. 6, the z-axis (or the yaw axis) is based on the direction of gravity. The x-axis (or the roll axis) and the y-axis (or the pitch axis) can be chosen relatively arbitrarily as long as the three axes are perpendicular to each other.

FIGS. 7A-7D illustrate an inertial frame of reference 700 for a wearable device according to some embodiments of the present disclosure. FIG. 7A depicts the inertial frame of reference 00 for the wearable device in a context where a user is swimming. In FIG. 7A, the user wears the wearable device 100, but the z-axis (or the yaw axis) in the inertial frame of reference 700 is based on the direction of gravity rather than the wearable device itself. In some embodiments, the x-axis (or the roll axis) and the y-axis (or the pitch axis) can be chosen relatively arbitrarily as long as the three axes are perpendicular to each other. In FIG. 7A, the z-axis is also referred to as yaw axis because any yaw movement rotates around the z-axis. Similarly, the x-axis is also referred to as roll axis because any roll movement rotates around the x-axis. And the y-axis is also referred to as pitch axis because any pitch movement rotates around the y-axis. By knowing the difference between the three-axes in the fixed-body frame of reference 500 and the three-axis in the inertial frame of reference 700 for the wearable device, the rotational data expressed in the fixed-body frame of reference 500 can be converted into the rotational data expressed in the inertial frame of reference 700 using techniques appreciated by people skilled in the art.

FIG. 7B illustrates an orientation of the wearable device 100 with respect to the inertial frame of reference 700. In FIG. 7B, a first device orientation 710 within the inertial frame of reference 700 has rotational data that include an angle (ϕ) 702 with respect to the positive x-axis, an angle (θ) 704 with respect to the positive y-axis, and an angle (ψ) 706 with respect to the positive z-axis. The first device orientation 710 can be expressed in an inertial frame of reference 700 as [cos(ϕ), cos(θ), cos(ψ)].

FIGS. 7C and 7D illustrate how the same device orientations shown in FIGS. 5C and 5D are expressed differently in inertial frame of reference 700. In FIG. 7C, wearable device 100 is held vertically in the same position as the wearable device in FIG. 5C. As discussed above in FIG. 6, the z-axis is based on the gravity in the inertial frame of reference 700. In FIG. 7C, the positive z-axis is chosen as the direct opposite position of gravity, the x-axis is perpendicular to the z-axis and pointing right horizontally, and the y-axis is perpendicular to both x-axis and y-axis and pointing “out” of the page of FIG. 7C. The fifth device orientation 550 in FIG. 7C aligns with the direction pointed by the crown 120. Accordingly, the fifth device orientation 550 has rotational data including the angle (ϕ) between the fifth device orientation 550 and the positive x-axis measuring 0-degrees; the angle (θ) between the fifth device orientation 550 and the positive y-axis measuring 90-degrees; and the angle (ψ) between the fifth device orientation 550 and the positive z-axis measuring 90-degrees. Therefore, the fifth device orientation 550 can be expressed as [cos(0), cos(90), cos(90)], which is [1, 0, 0]. In FIG. 7C, the fourth device orientation 540 represents direction of gravity and is parallel with and pointing toward the negative z-axis. The fourth device orientation 540 has rotational data including the angle (ϕ) between orientation 540 and the positive x-axis measuring 90-degrees; the angle (θ) between the fourth device orientation 540 and the positive y-axis measuring 90-degrees; and the angle (ψ) between the fourth device orientation 540 and the positive z-axis measuring 180-degrees. Therefore, the fourth device orientation 540 in FIG. 7C can be expressed as [cos(90), cos(90), cos(180)], which is [0, 0, −1].

In FIG. 7D, wearable device 100 is rotated 45-degree clockwise compared with FIG. 7C. The three axes are included in the inertial frame of reference 700 which is based on gravity. Therefore, the three axes remain in the same position as FIG. 7C because the inertial frame of reference 700 does not move with the wearable device. The fifth device orientation 550 in FIG. 7D represents the direction pointed by the crown 120. The fifth device orientation 550 corresponds to rotational data including the angle (ϕ) between the fifth device orientation 550 and the positive x-axis measuring 45-degrees; the angle (θ) between the fifth device orientation 550 and the positive y-axis measuring 90-degrees; and the angle (ψ) between the fifth device orientation 550 and the positive z-axis measuring 135-degrees. Therefore, the fifth device orientation 550 can be expressed as [cos(45), cos(90), cos(135)], which is [0.707, 0, −0.707]. As another example, the fourth device orientation 540 represents direction of gravity in FIG. 7D. The fourth device orientation 540 corresponds to rotational data including the angle (ϕ) between orientation 540 and the positive x-axis measuring 90-degrees; the angle (θ) between the fourth device orientation 540 and the positive y-axis measuring 90-degrees; and the angle (ψ) between the fourth device orientation 540 and the positive z-axis measuring 180-degrees. Therefore, the fourth device orientation 540 in FIG. 7D can be expressed as [cos(90), cos(90), cos(180)], which is [0, 0, −1].

It is noted that the fourth device orientation 540 is the same in FIG. 7C and FIG. 7D even though wearable device 100 has rotated. This is because the inertial frame of reference 700 is always fixed with respect to gravity. As a result, when position of wearable device 100 changes, the three axes in inertial frame of reference 700 do not move. On the other hand, the fifth device orientation 550 does move with respect to the three axes, so rotational data for the fifth device orientation 550 changes in the inertial frame of reference 700 even though it is fixed in body-fixed frame of reference 500.

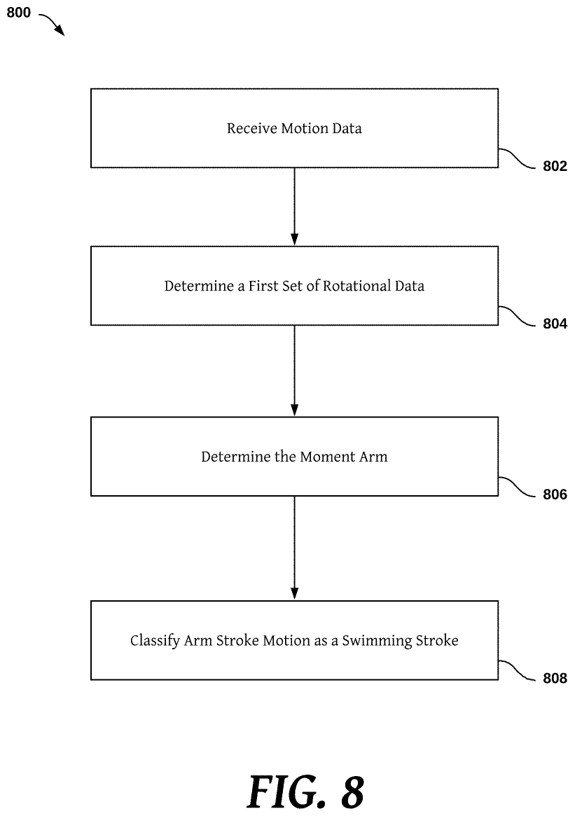

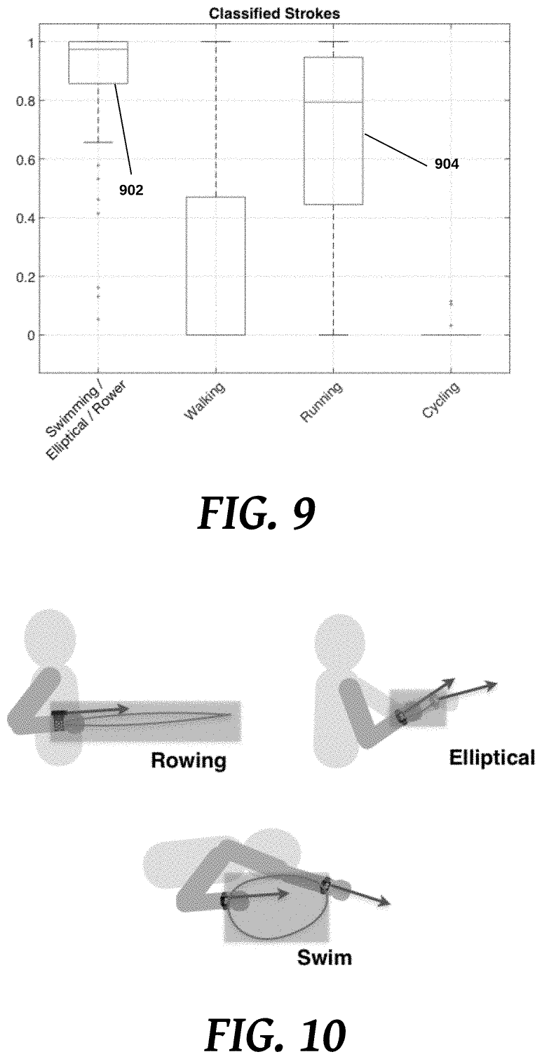

FIGS. 8-12 illustrate a method for classifying motion performed by a user as a swimming stroke according to various embodiments of the present disclosure. FIG. 8 shows a flow chart illustrating a process of classifying arm stroke motions (e.g., rowing arm stroke, elliptical arm stroke, running arm stroke, swimming arm stroke, and the like) performed by a user. FIG. 9 is a graph illustrating classification statistics for various arm stroke types commonly detected by the wearable device. As shown in FIG. 9, motion data for swimming, elliptical, and rowing arm strokes 902 is highly selective with a 98% true positive classification rate and 85% confidence. Motion data for running arm strokes 904 is less selective with an 80% true positive classification rate and only 50% confidence. Motion data for walking and cycling arm strokes was not consistent enough to produce a measureable true positive classification rate. This indicates motion data for walking and cycling arm strokes was too variable and/or unmeasurable to enable classification of walking and cycling activities using arm stroke motion.

FIG. 10 illustrates direction information and an exemplary stroke path for rowing, elliptical, and swim arm strokes. One or more features of these distinct motion patterns may be extracted from motion data to determine the type of activity performed by a user. In various embodiments, the direction of travel and/or angle relative to a fixed reference point (e.g., the horizon, gravity, the display surface of the wearable device, and the like) of the wearable device may be determined from orientation data, range of motion features, and/or angle features. FIG. 3 above illustrates an exemplary method for determining angle and range of motion features of a wearable device. FIGS. 5A-7D above illustrate exemplary device orientations determined from rotational data of a wearable device. Rotational data, device orientations, range of motion features, angle features, and/or other motion features for a particular arm stroke may be incorporated into stroke profile for an activity. For example, rotational data, device orientations, range of motion features and/or angle features observed in motion data captured during a swimming activity may be incorporated into a swimming stroke profile. In various embodiments, a motion classification model may classify arm stroke motions performed by a user as a particular type of activity (e.g., swimming activity, running activity, elliptical activity, and the like) by matching orientation data, range of motion features, angle features, and/or other motion features determined by surveying a plurality of datasets including motion data collected during an activity session having one or more motion features included in a particular stroke profile for the particular activity type. The plurality of datasets may include activities performed by the user and or a group of users having one or more characteristics (e.g., age, gender, weight, fitness level, and the like) in common with the user.

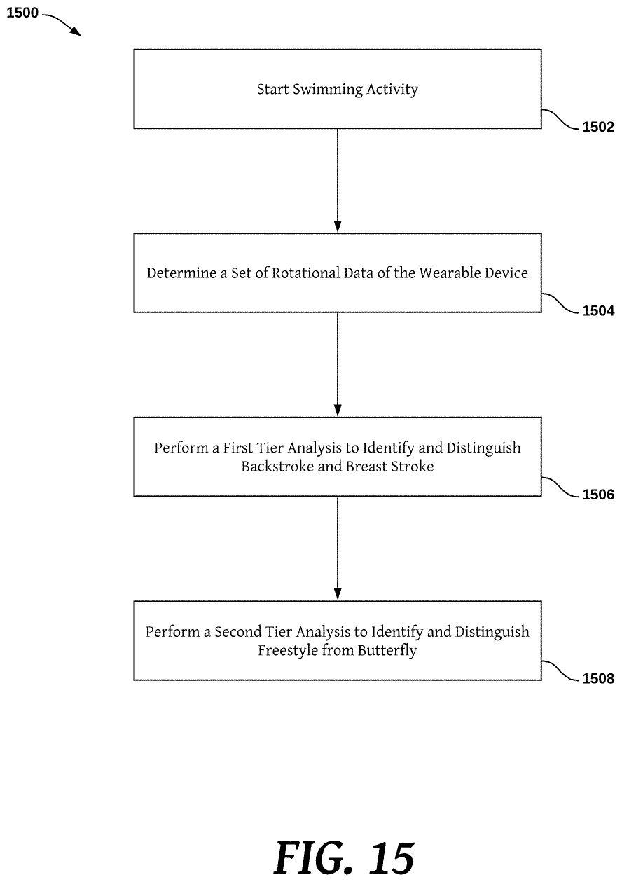

In various embodiments, the classification model may attempt to match motion data with a plurality of stroke profiles. The stroke profiles may be specific to a particular activity (e.g., running stroke profile, swimming stroke profile, elliptical stroke profile, rowing stroke profile, and the like). Stroke profiles may be assembled by surveying a plurality of datasets including motion data (e.g., 500 or more hours of activities including motion data) to extract motion features. The extracted motion features may then be standardized to establish global motion features for each activity type. Global motion features may include a subset of the motion features most frequently observed in the plurality of datasets including motion data for a particular activity. In various embodiments, stroke profiles may be personalized to particular user by extracting motion features from a plurality of datasets include motion data generated during activities performed only by the user and/or other users having one or more characteristics in common with the user. As shown in FIGS. 15-16E, stroke profiles may be assembled for specific swimming stroke types to identify swimming strokes performed during swimming activities.

In various embodiments, the classification model may be a machine learning system trained on a plurality of datasets including motion data captured during activity sessions performed while wearing the wearable device. During training, the plurality of datasets may be assembled into a training dataset that is particular to a specific activity type, stroke profile, motion feature, user, group of users, and the like. The machine learning classification model may identify motion features for a particular activity, stroke profile, user, etc. from motion data included in a training dataset. The machine learning system may then use the identified motion features to classify motion data included in an activity session that is not included in the training dataset.

The machine learning system may re-trained on additional datasets to improve classification accuracy by re-training the machine learning system using the motion data from the additional datasets to determine additional motion features and or adjust the weights of known motion features to weight the motion features that are stronger indicators of a particular activity or motion higher and weight the motion features that are weaker indicators of a particular activity or motion lower. To improve classification accuracy, the machine learning system may also be tuned by adjusting one or more hyperparameters (training algorithm, training time, training data, training cycles, and the like).

FIG. 8 illustrates an exemplary motion classification method 800. In some embodiments, the motion classification process can be modified by, for example, having steps combined, divided, rearranged, changed, added, and/or removed. To classify the motion of a user during an activity the wearable device may receive motion data from one or more motion sensors at step 802. At step 804, the wearable device 100 determines a first set of three dimensional rotational data of the wearable device 100 based on the motion data. Rotational data can include rotational movement used to determine the device orientations described above in FIGS. 5A-7D as well as angular velocity and angular acceleration.

Angular velocity can be expressed by Eq. 1 below:

ω=[rad/s] Eq. 1.

Angular acceleration can be represented by Eq. 2 below:

α=Δω/Δt Eq. 2.

In some embodiments, the rotational data is received from gyroscope 232 (e.g., a three axes gyroscope that measures rotational data in three dimensions) and is expressed in a body-fixed frame of reference with respect to wearable device 100 and or an inertial frame of reference.

The motion information can also include acceleration measurements of wearable device 100 in up to three-dimensions. The acceleration measurements can be a combination of the radial and tangential acceleration and can be expressed by Eq. 3 below:

a=ω×(ω×r)+(α×r) Eq.3
r=moment arm

In some embodiments, the acceleration measurements are received from accelerometer 234 (e.g., a three-axes accelerometer that measures linear acceleration in three dimensions) and are expressed in a body-fixed frame of reference with respect to wearable device 100 and or an inertial frame of reference. In embodiments having rotational data that includes angular acceleration, the angular velocity and or angular position of the wearable device may be obtained by integrating the angular acceleration over time. In embodiments having rotational data that includes angular velocity, the angular position of the wearable device can be obtained by integrating the angular velocity over time.

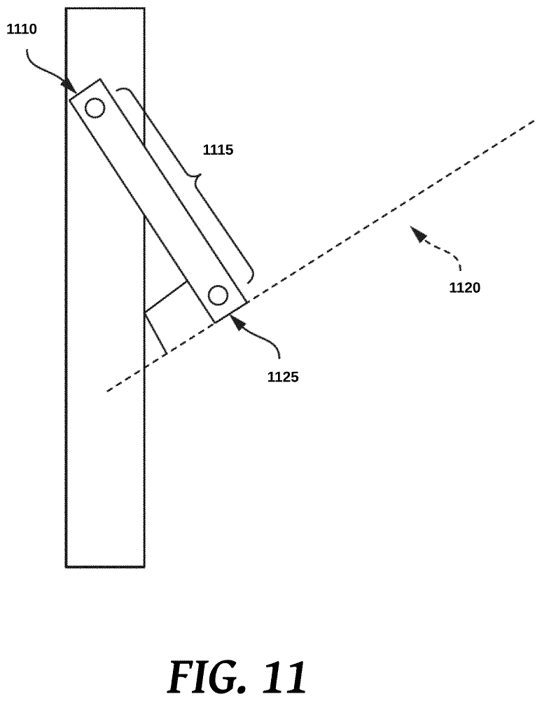

At step 806, based on the rotational data received from the gyroscope and the acceleration measurements received from the accelerometer, the moment arm can be computed. In some embodiments, for example as shown in FIG. 11, the moment arm 1115, computed by wearable device 1125, represents the extension of the arm from the shoulder joint 1110. As shown in FIG. 11, the moment arm 1115 is the perpendicular distance between the shoulder joint 1110 and the shoulder joint's line of force 1120. The line of force 1120 is tangential to the user's arm swing around the shoulder joint and is constantly changing direction.

In various embodiments, the moment arm 1115 is computed by taking the matrix representation of the cross product of a=ω×(ω×r)+(α×r) as shown in Eq. 3. The following is the computation of the cross product of acceleration (a) to find the moment arm, r:

a=WWr (where Wr represents the cross product of (ω×r))+Ur (where Ur represents the cross product of (α×r);

a=(WW+U)r

In various embodiments, r can be determined by solving the Least-Squares equation for r, for example, by using the Moore Penrose pseudoinverse.

The moment arm can be normalized (N) by taking several samples of accelerometer and gyroscope measurements and finding the average, which can be represented by the equations below:

aN=(WW+U)Nr

rN=(WW+U)N\aN

The computed length of the moment arm represents the user's arm extension and can be used to determine whether the user's arm swing is a swim stroke motion or an incidental movement. For example, a user's incidental arm swing generally rotates around the user's elbow joint or wrist, whereas the user's genuine swim stroke motion generally rotates around the user's shoulder. Therefore, an incidental arm swing will have a shorter moment arm length than a genuine stroke motion. As a result, the larger the moment arm length, the more likely the user's arm swing motion is a genuine swim stroke motion.

At step 808, based on the computed moment arm, the wearable device can classify the motion data generated by a user as a specific arm stroke motion (e.g., a swimming arm stroke). In various embodiments, the wearable device may determine if a user is performing a swimming stroke by comparing the computed moment arm length to a moment arm threshold. The moment arm length to use as the moment arm threshold may be determined by surveying a plurality of datasets including moment arm lengths determined from motion data collected during activities including known swimming strokes and or known incidental arm movements. The plurality of datasets used to determine the moment arm threshold may include activities performed by the user and or a group of users having one or more characteristics in common with the user. The moment arm length threshold may correspond to the minimum moment arm length of swim strokes and may be a motion feature included in a stroke profile. The wearable device may classify motion based on the moment arm by comparing the computed moment arm length to the moment arm threshold. For example, if the computed arm length is greater than the motion arm threshold for a swimming arm stroke, then the user's arm swing motion may be determined to be a swimming arm stroke. The moment arm threshold can be customized based on a user's gender, age, range of motion, activity experience level, fitness level, and/or other suitable characteristic. Moment arm length may be used to classify a swimming arm stroke motion as a particular type of swimming stroke. In various embodiments, swimming stroke types can include freestyle, butterfly, back stroke and breast stroke.

In various embodiments, a range of moment arm lengths may be associated with each arm stroke motion. The range of moment arm lengths may be a motion feature included in a stroke profile for a particular swimming stroke. For example, a swimming arm stroke may have a moment arm threshold of 25 cm and a range of 0-20 cm greater than the moment arm threshold and 0-20 cm less than the moment arm threshold. Each value within the range, may be associated with a different confidence level that corresponds to the likelihood the moment arm value was generated during a particular swimming stroke motion. For example, a swimming stroke motion profile may have a moment arm threshold of 25 cm and a range of 0-20 cm above and below threshold. Using this swimming stroke motion profile, a calculated moment arm of less than 5 cm is very likely not a swimming arm stroke and a moment arm greater than 25 cm is very likely a swimming arm stroke. Moment arms between 5-25 cm and between 25-45 cm are range for a swimming arm stroke and are likely a swimming arm stroke even though they are less than or exceed the moment arm threshold. The different confidence levels associated with each value within the range of moment arm lengths reflect the likelihood that motion data having each particular moment arm length value within the range was generated during a swimming stroke.

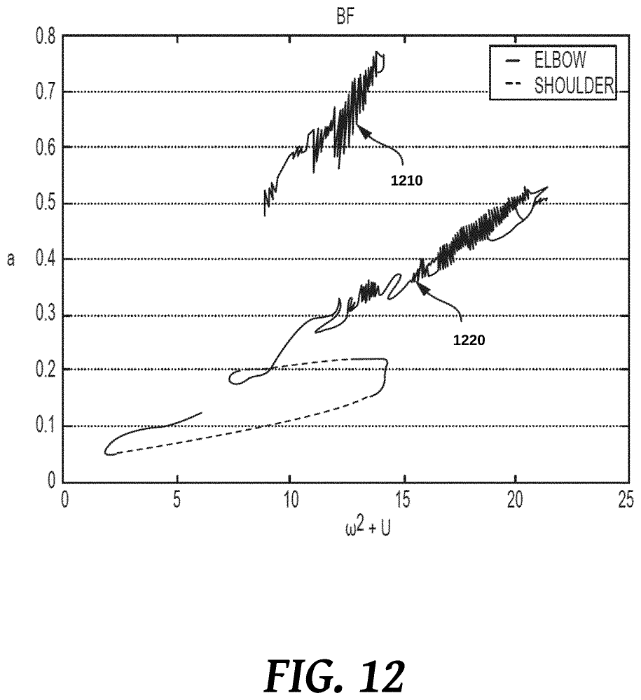

FIG. 12 illustrates a first set of rotational data, including acceleration data, of wearable device 100 during a period of time (e.g., 60 seconds) according to various embodiments of the present disclosure. FIG. 12 illustrates a first set of rotational data of wearable device 100 worn on a user's wrist during a swimming activity, and the first set of rotational data is expressed in the body-fixed frame of reference as described in connection with FIGS. 5A-5D. The x-axis represents WW+u and is measured in rad²/s², and the y-axis represents acceleration normalized by gravity and is measured in m/s².

The time period can be set by a user or the time period can be fixed. In some embodiments, the time period is proportional to a period that the user needs to complete several strokes. The wearable device 100 can dynamically set the time period based on average duration of user's strokes detected by wearable device 100. For example, if it takes a user three seconds to finish a stroke, then the time period can be set to nine seconds. In some embodiments, wearable device 100 can do sub-stroke measurements (e.g., 250 ms) or multi-stroke measurements (e.g., 6-9 seconds). A sub-stroke measurement tends to provide a near real-time measurement but can be a noisy estimate. While a multi-stroke measurement provides an “average” estimate of moment arm.

In the embodiment shown in FIG. 12, the rotational data, including acceleration data, is measured from two sessions of arm stroke motions: one session of arm strokes is rotating around the shoulder joint, as shown by the cluster of dots 1210 that appear at the top of the graph, and the other session of arm strokes is rotating around elbow joint, as shown by the cluster of dots 1220 that appear at the bottom of the graph. The slope of the data that is measured from the arm strokes around the shoulder joint is steeper than the slope of the data measured from the arm strokes around the elbow joint. In this embodiment, the steepness of the slope corresponds to the length of the moment arm. In other words, the steeper the slope, the greater the length of the moment arm. Typically, for a swim arm stroke, the moment arm length will be greater from the shoulder joint (as represented in FIG. 12 by the steeper slope of the cluster of dots 1210) than the elbow joint. If the rotation of the arm stroke occurs solely around the shoulder, then the moment arm is calculated from the wrist to the shoulder. If the rotation of the arm stroke occurs solely around the elbow, then the moment arm is calculated from wrist to elbow. If, however, the arm stroke motion is a combination of shoulder rotation and wrist rotation, then the combined stroke motion can provide an approximation of the moment arm of that combined motion.

In various embodiments, the wearable device 100 may determine a moment arm length threshold (i.e., a value or range of values for moment arm length) that is a characteristic of each of the different swimming stroke types. The moment arm length threshold for each swimming stroke type may be determined by surveying a plurality of datasets including motion data and or moment arm lengths measured during swimming activities having known stroke types. The wearable device can compare the computed moment arm length with the moment arm length value for each swimming stroke type to determine the type of swimming stroke performed by the user. The moment arm length threshold for each of the different swimming stroke types can be customized for a particular user based on gender, age, swimming level and/or other suitable characteristics. In some embodiments, the plurality of datasets used to determine the moment arm length threshold for each swimming stroke type include activities performed by the particular user or group of users having one or more characteristics in common with the user. FIGS. 15-16E below describe how the wearable device may classify swimming stroke types using moment arm length and other aspects of motion data. FIG. 16A shows exemplary moment arm lengths that are classified as the breaststroke swimming stroke type and FIG. 16B shows exemplary moment arm lengths that are classified as the freestyle swimming stroke type.

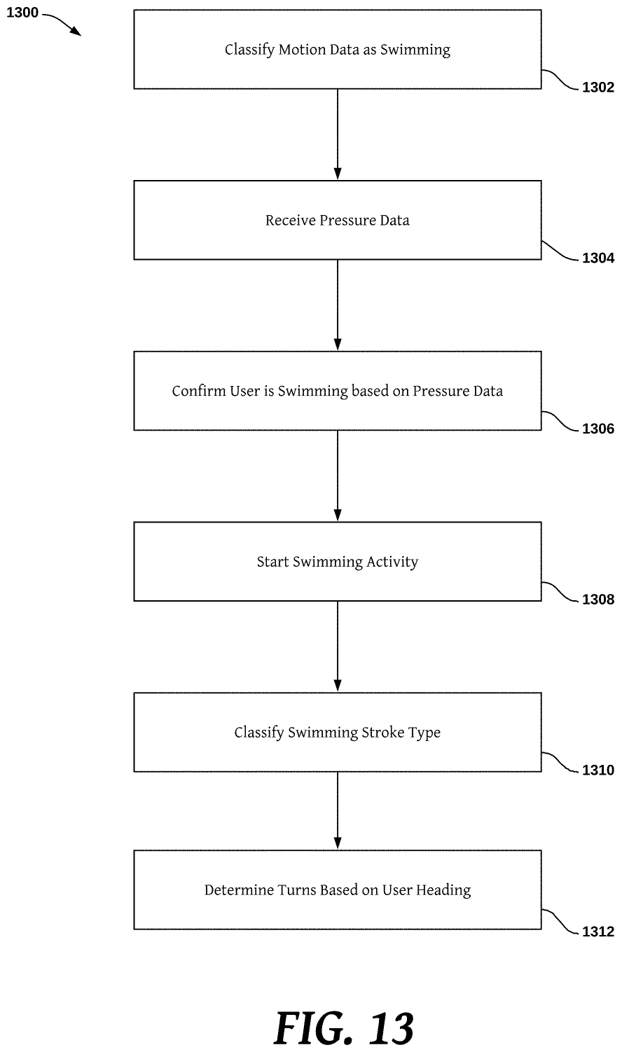

To improve the accuracy and precision of detecting swimming activities, the wearable device may use pressure data to confirm swimming activity motion classifications based on motion data. FIG. 13 illustrates an exemplary process 1300 for determining the start of a swimming activity. At step 1302, the wearable device detects a swimming activity by classifying motion data received from the motion sensors as a swimming motion. To classify the motion data, the wearable device may first detect a repeating pattern of user motion within the motion data. The pattern of user motion that periodically repeats (i.e., repeats on regular time intervals e.g., every 1-3 seconds) or may be interpreted by the wearable device as a user arm swing. Once the user's arm swing is detected the wearable device may classify the user's arm swing to determine the type of activity performed by the user. For example, the wearable device may classify motion data as swimming by determining the user's arm swing motion is a swimming stroke motion.

To minimize false positives, the wearable device may classify motion data as swimming upon detecting a swimming arm stroke or other swimming motion for a predetermined period of time (e.g., a fundamental period of approximately 6-9 seconds that includes two or more arm strokes or any other period of time long enough to capture multiple swimming strokes). In various embodiments, the wearable device may classify an arm stroke as a swimming stroke using moment arm lengths as described above. The wearable device may also classify motion data as swimming using the motion classification model described above in FIG. 3. The motion classification model may recognize one or more swimming motion features (e.g., moment arm length, the stroke rate, one or more wrist poses of the user, orientation data or other features generated based on rotational data, one or more range of motion features, and/or angle features) included in a swimming stroke profile within the motion classification model. The swimming motion features may be determined by surveying a plurality of datasets including motion data collected during known swimming activities and or known activities of other types (e.g., walking, cycling, standing, and the like). The plurality of datasets used to determine the swimming motion features may be performed by a particular user and or group of users having one or more characteristics in common with the particular user. Motion data included in the plurality of datasets may also be normalized to be consistent with motion data generated by the particular user.

Upon classifying motion data as swimming, the wearable device may confirm a user is swimming based on pressure data at step 1306. If the wearable device confirms the user is swimming, the wearable device may start a swimming activity at step 1308 to measure with user's energy expenditure during the swimming activity and one or more other swimming metrics (e.g., turns, breaths, laps, swim strokes, swim stroke styles, and the like). At step 1304, pressure data is received from a pressure sensor of the wearable device. To facilitate analysis, a plurality of pressure signals may be sampled from the pressure data for a predetermined period of time at a defined sample rate. At step 1306, the wearable device may confirm a user is performing a swimming activity based on pressure data. For example, the wearable device may compare the plurality of pressure signals extracted from the pressure data received from the one or more pressure sensors to a high pressure threshold (e.g., >500 kPa). If at least one of the measured pressure signals exceeds the high pressure threshold, the wearable device may determine the wearable device is submerged in water during the swimming activity and may confirm the user is swimming. If at least one of the measured pressure signals does not exceed the high pressure threshold, the wearable device may determine the wearable device is not submerged in water (i.e., the wearable device has some residual water on the surface, the wearable device made contact with water but was not submerged, and the like). In response to determining the measured pressure is below the high pressure threshold, the wearable device may confirm the user is not swimming and may avoid starting a swimming activity.

During certain swimming strokes, the wearable device is only submerged in water for a portion of the stroke. Therefore, the wearable device may continuously compare the measured pressure signals to the high pressure threshold for a predetermined period of time. If at least one of the measured pressure signals exceeds the high pressure threshold at any point during the predetermined period of time, the wearable device may confirm the user is swimming. If the measured pressure signals do not exceed the high pressure threshold at any point during the predetermined period of time, the wearable device may confirm the user is not swimming. The predetermined period of time corresponding to the period of one or more swimming strokes and or the high pressure threshold may be determined by surveying a plurality of datasets including pressure data, motion data, and timing information collected during known swimming activities and known non-swimming activities.

At step 1308, in response to confirming a swimming activity based on using pressure data, the wearable device may start a swimming activity. Upon starting a swimming activity the wearable device may classify the user's swimming stroke type at step 1310 and determine turns and other swimming metrics for the swimming activity at step 1312. At step 1310 the wearable device may classify a swimming stroke type (e.g., freestyle, breaststroke, backstroke, butterfly) based on motion data including rotational data, moment arm length, and the like. Rotational data and device orientations determined from rotational data may also be used to determine turns and other swimming metrics for the swimming activity.

To determine turns performed by a user during a swimming activity, the wearable device may determine a user heading (i.e., a direction of travel) based on rotational data. To determine a user heading, the wearable device may project the three dimensional (3D) rotation data discussed above in connection with FIGS. 5A-7D and shown below in FIG. 16C into a two dimensional (2D) vector. The 2D vector may then be filtered to reduce noise. For example, the three dimensional rotational data shown in FIG. 16C is a 3D vector that moves in time and can be represented as i(t)=(x(t), y(t), z(t)). Then, in some embodiments, i(t) can be projected onto the x-y plane using the gravity vector, and the resulting 2D vector can be represented as j(t)=(x(t), y(t)). The x-component and y-component of j(t) may each individually filtered by a low-pass filter. The relative heading calculated for the user (i.e., user heading) corresponds to the angle between j(t) (i.e., the 2D rotational motion vector). To detect changes in the user's direction, the relative heading may be plotted to show the user's heading at multiple time points during the swimming session. The change in the user's heading may then be calculated at adjacent times to show how j(t) is progressing in time. For example, suppose at t=0, (x=1, y=0), and then at t=1, (x=0, y=1), then the angle change (i.e., the change in heading) would be 90 degrees.

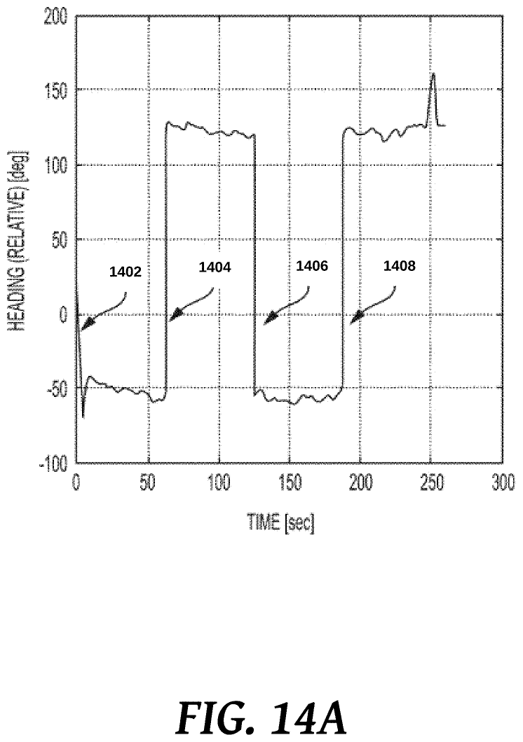

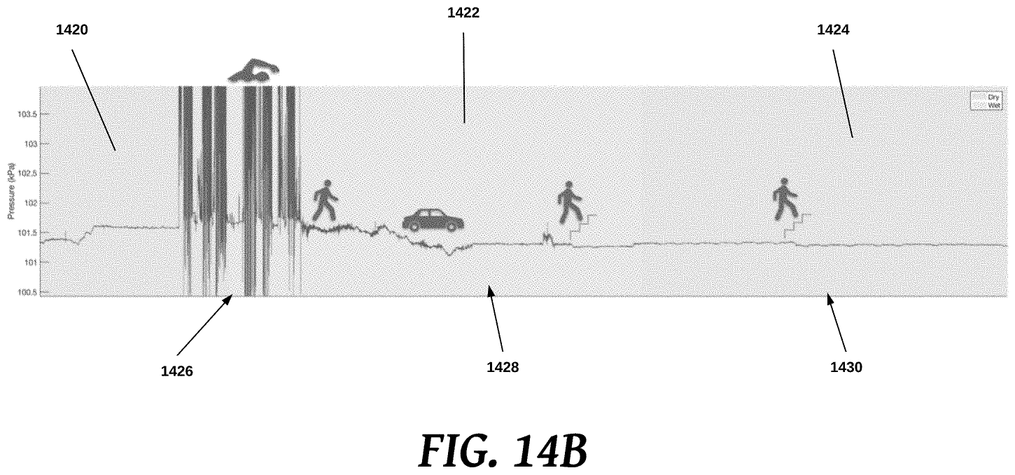

FIG. 14A shows the yaw data of wearable device 100 worn by a user who completes 4 laps in breaststroke. As described earlier, in an inertial frame of reference, the x-axis and y-axis can be chosen relatively arbitrarily as long as the three axes are perpendicular to each other. Therefore, the filtered yaw data of wearable device 100 in one direction can be around a relatively arbitrary value. For example, FIG. 14A shows the filtered raw data oscillates between two steady-state values, which are roughly 130 degrees and −50 degrees. The absolute values of the two steady-state yaw data (e.g., 130 degrees and −50 degrees) are not important; what is more important is that the two steady-state yaw data differ by approximately 180 degrees, which implies the user is making a turn. In FIG. 14A, the filtered raw data changes abruptly at 1402, 1404, 1406, and 1408 (for example, from 130 degrees to −50 degrees and/or from −50 degrees to 130 degrees) when the user is making a turn, and wearable device 100 can detect this abrupt change in heading and determine that the user is making a turn. In some embodiments, to detect a turn the change in heading may be compared to a threshold change in heading within a threshold time period. For example, in some embodiments, if the rotational data indicates a change in heading of more than 150 degrees per eight seconds, then wearable device 100 can determine that the user is making a turn. In some embodiments, other suitable threshold changes and/or threshold periods can be used. The change in heading and or time period included in the change in heading threshold can be determined by surveying a plurality of datasets including rotational data, user relative headings, and or changes in heading measured during swimming activities including one or more known turns performed during swimming. The swimming activities may be performed by the user of the wearable device and or a group of users having one or more characteristics in common with the user. The motion data included in the plurality of datasets may also be normalized to be consisted with motion data generated during swimming activities performed by the user.

The user heading may also be used to confirm the end of a swimming activity. In some swimming activities, the user is swimming laps in a pool. Each time the user completes a lap, the user performs a turn which results in a change in user heading. Therefore, swimming activities may include periodic changes in user heading. To confirm the end of a swimming activity, the wearable device may calculate a user heading and a change in user heading at multiple points during the swimming activity. The user heading and the change in user heading may be calculated based on rotational data and or motion data as described above. If the user heading changes on a periodic basis (i.e., changes between 150 degrees and −30 degrees every 8 seconds as described above), the wearable device may determine the user is swimming laps and may confirm the start of a swimming activity. If the user heading does not change on a period basis (i.e., does not change significantly or changes at irregular intervals, e.g., changes by more than 150 degrees in 2 seconds, then changes 110 degrees in 7 seconds, and the like) the wearable device may determine the user is not swimming and may confirm the end of the swimming activity.

FIG. 14B illustrates an exemplary graph including pressure data measured while the wearable device was wet (i.e., while the wearable device was in contact with water) and while the wearable device was dry (i.e., with no water contact). The beginning portion 1420 and end portion 1424 of the pressure data sample include pressure data captured while the wearable device was dray. The middle portion 1422 of the pressure data sample includes pressure data captured while the device was wet. The high pressure region 1426 illustrates exemplary pressure data that exceeds a high pressure threshold (e.g., 103 kPa). High pressure data as shown in the high pressure region 1426 detected while the wearable device is wet indicates the wearable device is submerged in water and that the user is swimming. The low pressure wet zone 1428 illustrates pressure data that may be observed when the wearable device is wet but not submerged in water (e.g., during a shower, walking in a rainstorm, and/or driving in the rain with the window open). The pressure data shown in the low pressure wet zone 1428, does not exceed a high pressure threshold, therefore indicates the wearable device is not submerged and that the user is not swimming. The low pressure dry zone 1430 illustrates pressure data that may be observed when the wearable device is dry. The pressure data shown in the low pressure dry zone 1430 does not exceed a high pressure threshold, therefore indicates the wearable device is not submerged in water and that the user is not swimming.

In various embodiments, the present disclosure describes a wearable device that may be configured to detect a swimming motion and in response to detecting a swimming motion, classify a user's swimming stroke into one of four common styles, including, freestyle, backstroke, breaststroke, and butterfly. In various embodiments, swimming motion can be classified into specific swimming stroke types according to techniques described in: U.S. patent application Ser. No. 15/691,245, filed on Aug. 30, 2017, and entitled “SYSTEMS AND METHODS FOR DETERMINING SWIMMING METRICS,” which patent application is incorporated herein in its entirety; U.S. patent application Ser. No. 15/692,726, filed on Aug. 31, 2017, and entitled “SYSTEMS AND METHODS OF SWIMMING ANALYSIS,” which patent application is incorporated herein in its entirety; and U.S. patent application Ser. No. 15/692,237, filed on Aug. 31, 2017, and entitled “SYSTEMS AND METHODS OF SWIMMING CALORIMETRY,” which patent application is incorporated herein in its entirety.

FIG. 15 shows a flow chart illustrating a method 1500 for classifying a user's swimming stroke style, according to various embodiments of the present disclosure. In some embodiments, the method 1500 of classifying a user's swimming stroke style can be modified by, for example, having steps combined, divided, rearranged, changed, added, and/or removed.

At step 1502, the wearable device 100 starts a swimming activity. As described above, the wearable device may start a swimming activity in response to detecting a swimming motion of a predefined period of time and or detecting pressure data that exceeds a high pressure threshold. The wearable device may detect a swimming motion based on samples of motion data output from one or more motion sensors 230. In some embodiments, the motion data can include any combination of gravity, acceleration, rotation, and or altitude. Based on the sampled motion data output from motion sensors 230, a fundamental period can be calculated. For example, information from the one or more motion sensors 230 can be sampled at 14 Hz. The fundamental period may include motion data for a period equivalent to two or more strokes. In various embodiments, the wearable device may determine a stroke rate from the motion data. The stroke rate may be used to determine a time period that includes motion data for two strokes. In some embodiments, if the sampled data does not show a sufficiently periodic signal, then the wearable device may resample the motion sensor information until it receives a sufficiently periodic signal. The process for classifying a user's stroke can be performed on a per stroke basis in real time (i.e., in fractions of a second. The stroke classifications can also be reported to a user in real time and or after the user completes a lap or some other defined period of swimming. At step 1504, the wearable device 100 determines a set of rotational data based on the motion data measured by the one or more motion sensors of the wearable device. In some embodiments, the rotational data may include the angular position, angular velocity, and or angular acceleration of the wearable device, with respect to a frame of reference. In some embodiments, if the rotational data of wearable device 100 is angular acceleration, then angular velocity and/or angular position can be obtained by integrating the angular acceleration over time. Likewise, if the rotational data of wearable device 100 is angular velocity, then angular position can be obtained by integrating the angular velocity over time. In some embodiments, the set of rotational data is received from gyroscope 232 and is expressed in a body-fixed frame of reference with respect to wearable device 100. In some embodiments, the acceleration data is received from accelerometer 234 and is also expressed in a body-fixed frame of reference with respect to wearable device 100.

FIGS. 16C-E illustrate exemplary rotational data of a wearable device during a swimming activity. FIG. 16C shows a series of graphs 1610, 1620, 1630, 1640, that depict exemplary 3D rotational data of the wearable device 100, as worn by a user during a swimming activity. Specifically, each graph corresponds to one of the four swim stroke styles (i.e., graph 1610 corresponds to freestyle, graph 1620 corresponds to backstroke, graph 1630 corresponds to breaststroke and graph 1640 corresponds to butterfly) and depicts the 3D rotational data of the wearable device for 30 strokes of that stroke style. Each graph includes three axes: an axis that represents the orientation of the face of the wearable device, an axis that represents the orientation of the crown of the wearable device, and an axis that represents the orientation of the band of the wearable device. Each axis ranges from 1, which represents pointing down to the ground, to −1, which represents pointing up towards the sky. As indicated by graphs 1610, 1620, 1630, and 1640, both breaststroke (graph 163-0) and backstroke (graph 1620) exhibit unique orbits that make them easy to differentiate from freestyle (graph 1610) and butterfly (graph 1640). However, freestyle and butterfly exhibit similar 3D rotational that make them more difficult to distinguish from each other. Accordingly, in various embodiments of the disclosed subject matter, a two tier analysis can be performed. During the first tier of analysis at step 1508, features are extracted from the set of rotational data to identify breaststroke and backstroke and distinguish these stroke styles from butterfly and freestyle. If the stroke is identified as breaststroke or backstroke, then a second tier of analysis does not have to be performed. Otherwise, if breaststroke and backstroke are ruled out, then a second tier analysis can be performed on the set of rotational data at step 1508. The second tier analysis may identify whether the stroke is freestyle or butterfly. In some embodiments, a second tier analysis can be performed regardless of the results of the first tier analysis.

At step 1506, a first tier analysis can be performed by analyzing certain features from the set of rotational data to identify backstroke and breaststroke and distinguish these stroke styles from butterfly and freestyle. According to some embodiments of the disclosed subject matter, at least three features can be used to identify backstroke and breaststroke and distinguish these stroke styles from butterfly and freestyle. These three features can include (1) mean crown orientation during the fastest part of user's stroke; (2) correlation of user's arm and wrist rotation; and (3) how much rotation about the crown contributes to the total angular velocity. These foregoing features are not intended to differentiate freestyle from butterfly.

According to some embodiments, as depicted by the graph 1650 in FIG. 16D, two of the three features of the first tier analysis are graphed for each of the different swim styles. Specifically, the y-axis 1660 represents the correlation of the arm and wrist rotation during the fastest part of the stroke, ranging from −1 (negative correlation, where the wrist and arm rotate in different directions), 0 (no correlation) to 1 (positive correlation, where the wrist and arm rotate in the same direction). As shown in the upper left portion of the graph, the backstroke exhibits a positive correlation of the arm and wrist rotations (i.e., the wrist rotates inward, then the arm rotates downward), while the breaststroke exhibits negative correlation of the arm and wrist rotations (i.e., the wrist rotates outward, then the arm rotates downward). Further, the x-axis 1662 of graph 1650, represents the mean crown orientation of the wearable device (which is a proxy for the orientation of a user's fingertips) during the fastest part of the stroke, ranging from −1, where user's fingertips (or the crown) faces up towards the sky, to 1, where the user's fingertips (or crown) is oriented downwards, facing the earth. As depicted in graph 1650, during the fastest part of the backstroke 1652 (i.e., during the recovery phase when the hand is out of the water and making an arc towards the sky), the user's fingertips face upwards towards the sky, while breaststroke 1658, the user's fingertips face downwards towards the earth when the hand is moving fastest.

Also shown in graph 1650, in FIG. 16D, the butterfly 1656 and freestyle 1654 strokes exhibit similar correlation between arm and wrist rotation (i.e., both exhibit a positive correlation of the arm and wrist rotations), as well as similar crown orientations during the fastest part of the strokes (i.e., fingertips facing downwards towards the earth), making these strokes difficult to distinguish from each other based on these two features. In contrast, the backstroke is easily distinguishable based on (1) a negative arm-wrist correlation and (2) the mean crown orientation facing up towards the sky during the fastest part of the stroke. The breaststroke is also easily distinguishable based on (1) a positive arm-wrist correlation and (2) the mean crown orientation facing downwards during the fastest part of the stroke.

The next series of graphs shown in FIG. 16E, focus on the mean crown orientation feature, discussed above in connection with FIG. 16D. Specifically, the series of graphs shown in FIG. 16E, depict the mean crown orientation with respect to gravity, weighted by the faster parts of the stroke. This feature is a proxy for the direction that the user's fingertips are pointing when the user's arm is moving the fastest. The mean crown orientation feature can be expressed by the following equation:

mean_gx_w1=sum(gravity_x*total_user_acceleration)/sum(total_user_acceleration) Eq. 4.

The series of graphs depicted in FIG. 16E, correspond to the crown orientation for each of the different swim stroke styles (i.e., graph 1654 corresponds to freestyle, graph 1658 corresponds to breaststroke, graph 1652 corresponds to backstroke and graph 1656 corresponds to butterfly). The y-axis of each of the graphs represents crown orientation z, where −1=crown facing up towards the sky, 0=the crown facing parallel to the horizon, and 1=crown facing down towards the earth. The x-axis of each of the graphs represents time in seconds.

The crown orientation feature can be used to identify backstroke and breaststroke and distinguish these stroke styles from the other swim stroke styles. As shown in graph 1665, the user's fingertips in backstroke trace an arc from the horizon to the sky and back to horizon, when the user's arm is out of the water and moving fast. Unlike the other swim stroke styles, the orientation of the crown in backstroke is above the horizon for half the stroke and faces the sky during points of high acceleration.

For breaststroke, as depicted in graph 1664, the crown goes above the horizon during the quiescent portions of the stroke and faces downward during the fastest parts of the stroke. For both freestyle (graph 1662) and butterfly (graph 1668), the crown rarely goes above the horizon and faces parallel to the horizon during the fastest parts of these strokes, making these strokes hard to distinguish from each other based on this feature.

At step 1510, after a first tier analysis is performed on the set of rotational data, a second tier analysis can be performed to distinguish freestyle from butterfly. In some embodiments, nine features can be used during the second tier analysis to distinguish between butterfly and freestyle.

A first feature that can be used is relative arm rotation about the band during the pull phase, which can be expressed by the following equation:

RMS(rotation y during pull phase)/RMS(rotation y during entire stroke), where RMS is root mean square Eq. 5.

The ratio for the relative arm rotation features tends to be higher for butterfly, because butterfly, in comparison to freestyle, tends to have more (stronger) rotation around the band of wearable device 100 during the pull phase, but similar or less rotation around the band during the recovery phase. During the recovery phase of butterfly, the palms tend to stay more parallel to the horizon than during freestyle which results in less rotation about the band during recovery. Since the hands are more parallel during recovery in butterfly, the rotation tends to be around the face (less rotation around the band). For freestyle, the hands are less parallel so there is more rotation around the band.

A second feature that can be used is the moment arm feature range(uxz)/range(wy), where:

uxz=sqrt(sum(user_x2+user_z2), wy=abs(rotation_y), range(x)=max(x)−min(x) Eq. 6.

The moment arm feature captures the longer moment arm (i.e., arms outstretched) during butterfly, in comparison to freestyle. This feature compares rotation around the band (i.e., axis y) to the linear acceleration in the plane perpendicular to the band. The longer the moment arm, the more linear acceleration relative to rotation there will be.

A third feature that can be used to distinguish butterfly from freestyle is the ratio of acceleration z to rotation y. This is another version of moment arm and can be expressed by:

uz/wy, where uz=sum(abs(rotation_y)),uz+sum(abs(user_z)) Eq. 7.

A fourth feature that can be used to distinguish butterfly from freestyle is mean gravity crown weighted by acceleration, similar to the feature used during the first tier analysis, discussed above in connection with FIGS. 16C-16E. This feature measures the orientation of the crown (which is a proxy for the orientation of user's fingertips during the stroke). It is weighted by the faster parts of the stroke to give more weight to the recovery phase of the stroke. In butterfly, the crown orientation with respect to gravity is close to zero, which captures that the user's hands stay more parallel to the horizon during butterfly, in comparison to freestyle.

A fifth feature that can be used to distinguish butterfly from freestyle is the correlation between gravity_y(top of band orientation) and rotation_y(rotation around the band) and can be measured by the equation:

$$r = \frac{{\sum }_{i = 1}^{n} ( {x}_{t} - \overline{x} ) ( y - \overline{y} )}{\sqrt{{\sum }_{i = 1}^{n} {( {x}_{t} - \overline{x} )}^{2}} \sqrt{{\sum }_{i = 1}^{n} {( y - \overline{y} )}^{2}}} . \qquad Eq . 8$$

Specifically, this feature measures how the wrist and arm rotate together during the stroke. The wrist and arm correlation is lower for butterfly than freestyle, indicating that there are more times during the butterfly stroke where the arm is rotating, but the wrist is not. This feature also captures that the hands stay more parallel to the horizon during butterfly (i.e., arms swing around with less wrist rotation), in comparison to freestyle.

A sixth feature that can be used to distinguish butterfly from freestyle is RMS of crown rotation, which can be expressed by the equation:

RMS(rotation_x) Eq. 9.

This feature captures the stronger rotational energy exhibited by butterfly, in comparison to freestyle.

A seventh feature that can be used to distinguish butterfly from freestyle is minimum rotation around the crown, which can be expressed by the equation:

min(rotation_x) Eq. 10.

This feature also captures the stronger rotational energy exhibited by butterfly, in comparison to freestyle.

An eighth feature that can be used to distinguish butterfly from freestyle is maximum rotation around the band, which can be expressed by the equation:

max(rotation_y) Eq. 11.

This feature also captures the stronger rotational energy exhibited by butterfly, in comparison to freestyle.

A ninth feature that can be used to distinguish butterfly from freestyle is maximum rotation_x over y, which can be expressed by the equation:

max(abs(rotation_x)/max(abs(rotation_y)) Eq. 12.

This feature also captures the stronger rotational energy exhibited by butterfly, in comparison to freestyle.

These nine features can be used together in a two-way logistic regression to distinguish butterfly from freestyle and can be weighted, based on their usefulness in distinguishing butterfly from freestyle. It is understood that most classifiers (SVM, LDA, etc.) will perform similarly with this same feature set. It is further understood that the nine features discussed above are exemplary, and other suitable features may be used as well. In some embodiments, the nine features of the second tier analysis, have the following order of usefulness, ranked from greatest to least:

| Rank | Feature |
|---|---|
| 1 | Relative arm rotation during the pull phase |
| 2 | Range ration of ZX acceleration to rotation y |
| 3 | Ratio of acceleration z to rotation y |
| 4 | Max. rotation around band |
| 5 | Max. rotation X over Y |
| 6 | Mean gravity crown weighted by acceleration |
| 7 | Correlation between gravity_y (top of band orientation) compared |
| | to rotation_y (rotation around band). |
| 8 | RMS of crown rotation |
| 9 | Min. rotation around crown |

To increase the accuracy of swimming activity detection and performance information calculated during swimming activities, the wearable device may request a user confirm the start and/or end of a swimming activity. For example, after detecting a swimming activity, the wearable device may send a request to a user to confirm the start of a swimming activity. After detecting the end of a swimming activity, the wearable device may send a request to a user to confirm the end of a swimming activity.

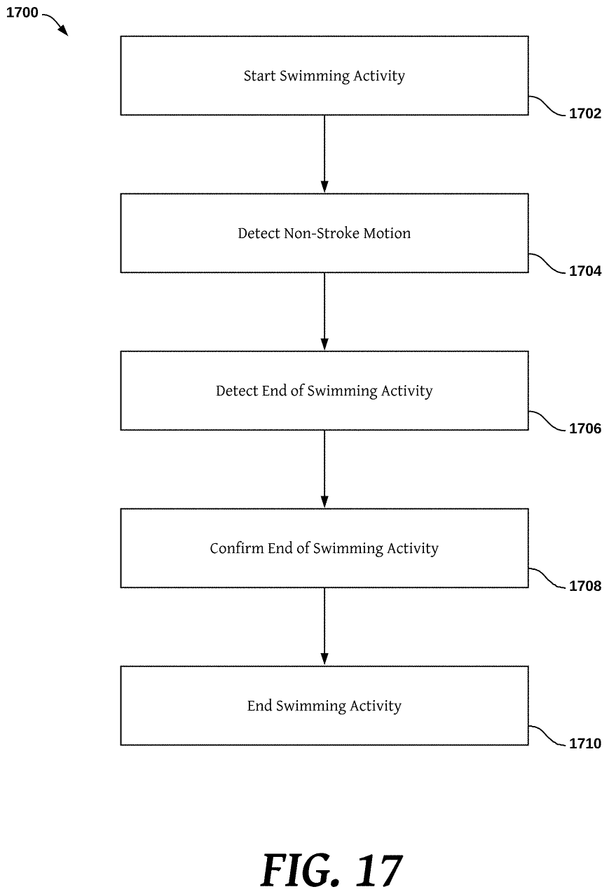

FIG. 17 illustrates an exemplary process 1700 for determining the end of a swimming activity. As described above, at step 1702 the wearable device may start a swimming activity upon detecting a swimming motion and or confirming the user is swimming based on pressure data and or user heading. At step 1704, the wearable device may determine the motion data is not a swimming motion. For example, the wearable device may classify the motion data received by the one or more sensors as a non-swimming motion based on moment arm lengths and or rotational data. The wearable device may also use a motion classification model to determine the motion data does include a swimming motion based on one or more angle features and or motion features. The angle features and or motion features that indicate the user is performing a non-swimming activity may be determined by surveying a plurality of datasets including motion data collected during known non-swimming activities. The activities included in the plurality of datasets may be performed by the user and or a group of users having one or more characteristics in common with the user. The motion data included in the plurality of datasets may also be normalized to be consistent with motion data generated by the user.

In response to detecting a non-swimming stroke motion for a predetermined period of time (e.g., 6-9 seconds or any other time period suitable to determine the user has stopped swimming), the wearable device may detect a stop and or the end of the swimming activity at step 1706. To differentiate between a temporary stop and the end of a swimming activity, the wearable device may confirm the end of the swimming activity at step 1708. For example, the wearable device may confirm the end of the swimming activity based on pressure data. If the wearable device detects a pressure signal that is below the high pressure threshold for swimming, the wearable device may determine the wearable device is not submerged in water and may confirm the end of the swimming activity. The wearable device may also compare the measured pressure signal to a dry pressure threshold that indicates the wearable device is dry. If the pressure signal is below the dry pressure threshold then the wearable device may confirm the end of the swimming activity. To increase detection accuracy, measured pressure data may be continuously compared to the high pressure threshold and or the dry pressure threshold for a predetermined period of time (e.g., 30 s). If, at any point during the predetermined time period, the pressure signal is above the high pressure threshold, the wearable device may determine the user is swimming. If, at any point during the predetermined time period, the pressure signal is below the dry pressure threshold, the wearable device may confirm the end of the swimming activity. The high pressure threshold, dry pressure threshold, and duration of the predetermined time period may be determined by surveying a plurality of datasets including pressure data captured during known transitions between swimming activities and other activity types.

The wearable device may also confirm the end of the swimming activity at step 1708 based on motion data. For example, the wearable device may identify a motion indicative of a non-swimming activity (i.e., motion that does not include a user arm swing). In response to detecting the non swimming motion, the wearable device may confirm the end of the swimming activity. The non-swimming motion may have no consistently repeating pattern and may be incidental motion while the user is stationary and/or performing an non-workout activity (e.g., driving, typing, reading, shopping, and the like). The wearable device may also confirm the end of the swimming activity based on user heading derived from rotational data. For example, if, the wearable device determines the user heading is not changing on a periodic basis, the wearable device may determine the user is not swimming and may confirm the end the swimming activity.

In response to confirming the end of the swimming activity, the wearable device may end the swimming activity at 1710. Once the swimming activity is ended, the wearable device may stop calculating performance information (i.e., a user exertion level) and or swimming metrics and may present the performance information and or swimming metrics calculated during the swimming activity to the user.

1. A method for improving performance of a wearable device while recording a swimming activity, the method comprising:
receiving motion data of a user from one or more motion sensors of the wearable device;
receiving pressure data from a pressure sensor of the wearable device;
detecting, by a processor circuit of the wearable device, a start of the swimming activity, the detecting the start of the swimming activity comprising:
determining, by the processor circuit using the motion data, rotational data expressed in a frame of reference based on the motion data;
classifying, by the processor circuit a user's arm swing as a swim stroke motion based on the rotational data and the motion data;
detecting, by the processor circuit, the swim stroke motion in the motion data for a first predetermined period of time; and
confirming, by processor circuit, the user is swimming based on the pressure data; and
determining, by the processor circuit one or more swimming metrics for the swimming activity in response to detecting the start of the swimming activity.

2. The method of claim 1, wherein the confirming the user is swimming based on the pressure data comprises:
sampling, by the processor circuit, a plurality of pressure signals from the pressure data;
continuously comparing, by the processor circuit, the plurality of pressure signals to a high pressure threshold; and
detecting, by the processor circuit, at least one pressure signal that exceeds the high pressure threshold.

3. The method of claim 1, further comprising:
detecting, by the processor circuit, an end of the swimming activity based on the motion data and the rotational data by:
determining, by the processor circuit, the user's arm swing does not include the swim stroke motion for a second predetermined period of time;
determining, by the processor circuit, a user heading at multiple time points during the swimming activity based on the rotational data;
continuously calculating, by the processor circuit, a change in user heading during the swimming activity; and
determining, by the processor circuit, the user heading is not changing on a periodic basis.

4. The method of claim 3, further comprising confirming the end of the swimming activity based on pressure data by;
sampling, by the processor circuit, a plurality of pressure signals from the pressure data;
continuously comparing, by the processor circuit, the plurality of pressure signals to a dry pressure threshold; and
detecting, by the processor circuit, at least one pressure signal included in the plurality of pressure signals is below the dry pressure threshold.

5. The method of claim 1, comprising:
determining, by the processor circuit, a user heading from the rotational data at a first time point and a second time point;
calculating, by the processor circuit, a change in device heading at the second time point relative to the first time point; and
comparing, by the processor circuit, the change in user heading to a change in heading threshold.

6. The method of claim 5, comprising in response to determining the change in user heading exceeds the change in heading threshold, determining, by the processor circuit, a user is performing a turn during a swimming activity.

7. The method of claim 5, comprising in response to determining the change in user heading is below the change in heading threshold, confirming, by the processor circuit, an end of the swimming activity.

8. The method of claim 1, wherein the classifying the user's arm swing as a swimming motion further comprises:
determining, by the processor circuit, a moment arm for the user's arm swing during a fundamental period;
comparing, by the processor circuit, the moment arm to a moment arm threshold; and
detecting, by the processor circuit, the moment arm exceeds the moment arm threshold at any point during the fundamental period.

9. The method of claim 1, wherein the classifying the user's arm swing as a swimming motion further comprises:
extracting, by the processor circuit, a first set of features from the motion data;
comparing, by the processor circuit, the first set of features to plurality of swimming motion features included in a motion classification model; and
matching, by the processor circuit, the first set of features with one or more features included in the plurality of swimming motion features.

10. The method of claim 9, wherein first set of features includes a period of time required to complete a stroke, one or more wrist poses of the user, and one or more motion features extracted from the rotational data.

11. The method of claim 1 comprising:
in response to detecting the start of the swimming activity, calculating, by the processor circuit, performance information of the user during the swimming activity, the performance information including a level of exertion based on a heart rate of the user measured by a heart rate sensor and the one or more swimming metrics.

12. The method of claim 1, wherein the one or more swimming metrics include turns, breaths, laps, swimming styles, and swimming strokes.

13. The method of claim 12, comprising:
in response to detecting the start of the swimming activity, outputting, by the processor circuit, the one or more swimming metrics on a display of the wearable device.

14. The method of claim 1, wherein the one or more motion sensors comprise at least one of an accelerometer and a gyroscope.

15. The method of claim 1, further comprising: classifying, by the processor circuit, a swim stroke type based on the motion data and the rotational data.

16. The method of claim 15, wherein the swim stroke type is at least one of a freestyle stroke, a breaststroke, a butterfly stroke, and a backstroke.

17. The method of claim 15 wherein the classifying a swim stroke type based on the motion data and rotational data comprises:
extracting, by the processor circuit, a second set of features from the rotational data; and
matching, by the processor circuit, the second set of features with one or more features included in a swimming stroke motion profile.

18. The method of claim 17, wherein the second set of features include an orientation of the wearable device, a device angle, a range of motion feature, a moment arm length a correlation of the user's arm and wrist rotation, mean crown orientation during the fastest part of the stroke, a ratio of acceleration long two or more axis of rotation, a minimum rotation relative to a frame of reference, and a maximum rotation relative to a frame of reference.

19. A system for improving performance of a wearable device while recording a swimming activity, the system comprising:
one or more motion sensors configured to collect motion data of a user;
a pressure sensor configured to collect pressure data; and
a processor circuit in communication with the one or more motion sensors and the pressure sensor, the processor circuit configured to execute instructions causing the processor circuit to:
determine rotational data expressed in a frame of reference based on the motion data;
detect a repeating pattern of user motion in the motion data;
classify a user's arm swing included in the repeating pattern of user motion as a swim stroke motion based on the motion data and the rotational data;
detect the swim stroke motion in the motion data for a first predetermined period of time; and
confirm the user is swimming based on pressure data.

20. The system of claim 19, wherein the processor circuit is further configured to:
sample a plurality of pressure signals from the pressure data;
continuously compare the plurality of pressure signals to a high pressure threshold;
detect at least one pressure signal that exceeds the high pressure threshold; and
confirm the user is swimming in response to detecting the at least one pressure signal that exceeds the high pressure threshold.
